SEQUENCE LISTING (40 sequences)

SEQ ID NO: 1 - protein, 751 aa, Zea mays
MALPLDSLLLHLTAPAPAPAPAPRRSHQTPTPPHSFLSPDAQVLVLAISSHPLPTLAAFLASRRDELLRADITSLLKALELSGHWEWALALLRWAGKEGAADASALEMVVRALGREGQHDAVCALLDETPLPPGSRLDVRAYTTVLHALSRAGRYERALELFAELRRQGVAPTLVTYNVVLDVYGRMGRSWPRIVALLDEMRAAGVEPDGFTASTVIAACCRDGLVDEAVAFFEDLKARGHAPCVVTYNALLQVFGKAGNYTEALRVLGEMEQNGCQPDAVTYNELAGTYARAGFFEEAARCLDTMASKGLLPNAFTYNTVMTAYGNVGKVDEALALFDQMKKTGFVPNVNTYNLVLGMLGKKSRFTVMLEMLGEMSRSGCTPNRVTWNTMLAVCGKRGMEDYVTRVLEGMRSCGVELSRDTYNTLIAAYGRCGSRTNAFKMYNEMTSAGFTPCITTYNALLNVLSRQGDWSTAQSIVSKMRTKGFKPNEQSYSLLLQCYAKGGNVAGIAAIENEVYGSGAVFPSWVILRTLVIANFKCRRLDGMETAFQEVKARGYNPDLVIFNSMLSIYAKNGMYSKATEVFDSIKRSGLSPDLITYNSLMDMYAKCSESWEAEKILNQLKCSQTMKPDVVSYNTVINGFCKQGLVKEAQRVLSEMVADGMAPCAVTYHTLVGGYSSLEMFSEAREVIGYMVQHGLKPMELTYRRVVESYCRAKRFEEARGFLSEVSETDLDFDKKALEAYIEDAQFGR

SEQ ID NO: 2 - DNA, 2257 bp, Zea mays
  source 1..2257: mol_type = genomic DNA
atggcgctcccgctcgactcgctcctgctccacctcaccgcgcccgcccccgcgccggcccccgcgccgcggcggtcgcaccagacgccgacgccgccgcactccttcctctcccccgacgcgcaggtgctggtgctcgccatctcctcgcacccgctccccacgctggcggccttcctggcctcccgccgcgacgagctcctccgcgcggacatcacgtccctgctcaaggcgctggagctctcggggcactgggagtgggcgctcgcgctcctccggtgggcaggcaaggagggtgccgccgacgcgtcggcgctcgagatggtcgtccgcgcgctgggccgcgagggccagcacgacgccgtctgcgcgctgctcgacgaaacgccgctcccgccgggctcccgcctcgacgtccgcgcctacaccaccgtgctgcacgcgctctcccgcgcgggccggtacgagcgcgcgctcgagctcttcgccgagctccggcgccagggggtggcgcccacgctcgtcacctacaacgtcgtgctggacgtgtacgggcggatgggccggtcgtggccgcggatcgtcgccctcctcgatgagatgcgcgccgccggggtcgagcccgacggcttcaccgccagcacggtgatcgccgcgtgctgccgcgacgggctggttgacgaggcggtggcgttcttcgaggacctcaaggcccgcggccacgccccgtgcgtcgtcacgtacaacgcgttgctccaggtgttcggcaaggccgggaactacacggaggcgctgcgcgtgctcggggagatggagcagaacggctgccagccagatgctgtgacgtacaacgagctcgccggaacgtacgcccgggctgggttcttcgaggaggctgccaggtgcctggacacaatggcatccaagggtctgttgccaaacgcattcacgtacaacaccgtgatgacagcctatgggaatgttgggaaggtggatgaggcgctcgctctgtttgaccagatgaagaagaccgggttcgtgccgaacgtgaacacgtacaatcttgtccttggcatgcttggcaagaagtcaaggttcacggtgatgctagagatgcttggagagatgtcgaggagcggatgcacaccgaaccgggtaacatggaacacaatgcttgcagtctgtgggaagcgtggcatggaggactacgtcacccgggttctggaggggatgaggtcttgcggggttgaactgagccgagacacctacaacaccctgatagctgcgtacggccggtgtggctcgaggactaatgccttcaagatgtacaacgagatgaccagcgctggattcaccccctgcatcaccacgtacaacgcgttgctgaacgtgctgtcgcggcagggcgactggtccaccgcccagtcgatcgtaagcaaaatgaggaccaaggggttcaagccgaacgagcagtcgtattcgctgctgctccagtgctacgcgaaggggggcaacgtggcagggatagccgcgatcgagaacgaggtgtacggatcaggtgccgttttcccaagctgggtgatcctgaggacccttgtcatcgccaatttcaagtgccggcgactggatggcatggagacggcgtttcaagaggtgaaggccagaggctacaacccggacctcgtgatattcaactccatgctgtccatctacgcgaagaacgggatgtacagcaaggccaccgaggtcttcgactccatcaagcggagcgggctgagccccgacctcatcacctacaacagcctgatggacatgtacgccaagtgcagcgagtcgtgggaggccgagaagatactgaaccagctcaagtgctcccagacgatgaagcccgacgtggtgtcctacaacacggtcataaacgggttctgcaagcaggggctggtgaaagaggcccagagggtcctctcggagatggtcgccgacggcatggccccctgcgccgtgacctaccacacgctcgtcgggggttactccagcctggagatgttcagcgaggccagggaggtcatcggctacatggtccagcacggcctcaagcctatggagctgacctacaggagagtcgtcgagagctactgcagagcgaagcggttcgaggaggctcgcggcttcctgtccgaggtctcggagaccgacctggattttgacaagaaggcgctcgaagcctatatagaggatgcgcagtttggaaggtaga

SEQ ID NO: 3 - DNA, 3540 bp, Zea mays
  source 1..3540: mol_type = genomic DNA
atccaagtgagttttacgggcccactggaccagaagcttctcaagctcaagcatttacttttctagttagagaccagcgtcttggagctaatgtagggtctgcccaaggaccgacgggtttaggtaaatatctaatgcgttccccaacgggagaggttatttttggaggggaaactatgcgtttttgggaccttcgtgctccgtggttagaacctctaaggggccccaacggtttggacttgagtaggttgaagaaagacggccaaccttggcaagaacgacgttcaggagaatatatgacccatgctcctttaggctctttaaattccgtgggtggcgtagctaccgagatcaatgctgttaattatgtctcccctagaagttggttagcgacctcccattttgttctaggattcttcttttttgtgggccatttgtggcatgcaggaagagcccgagctgctgcagcaggctttgaaaagggaattgatcgcgatttggaacctgttgtttacatgacccctcttaactaagattttttatttatacctgttctagtttttttctgttctggctcggttattccatctagccgagccattcatgcttttttatgaaagaatgatataaggggggctgcacaaagaaaaacatagatagaaagaagcaaatgtattcaatatacaaaaggagagagagggattcgaaccctcgatagttcctagaactataccggttttcaagaccggagctatcaaccactcagccatctctccacagcccaatccctattttattcctacaaatagaacatagctatatgaaatgatctactaactcatctcagatgcaagtccactttcaatatatctctgtataaagcggtaaggaaaaagttttaataaaaagaagaatcaatggattcatgattaaacccctcctacttcttgtattttcatacaattttgattaagtgagggatcaaatagaaatatgtagtcaactttatttgaaggtagtttggaggattataaatatgaatattgctttccaattagctgtttttgcattaattgcgacttcctcagttttagtaattagaggacaccttgtatttgcttctcctgatggttggtcaaataataaaaacgttgtattttccggtacatcattatggattggattagtctttctggtagctattctgaattctcttatttcttgaatttgtttagtatttagtaacccgatacaaaataaataaaaagaaaggccctttttcgaaaaaattaggatttttatacacattaaaatgctattttcgttccgaattattacctcttctctttcattattatgaaattctattgccattagaatattgattgatagacagttaataaaaagaaaactctaatagaaaatgaaacggtcgacccagacatagacggtcgacccagacatagacggtcgacccaggtggatataccctataaaatataggacgtagcaagcgtagttcaatgtagcgagcgtagttcagtggtaaaacatctccttgccaaggagaagatacgggttcgattcccgtcgctcgccagcttaatttagtaaggtgctatgataaaaaattcagtctagttgactacttaactacttaatattatagtttatatttaatattatagtttatattatagtaaattatatatatttatttttaatatagtaaattattatagtaaatgactacttaactacttctattaaattactatttaatattaaatgaatattaaattaagtagttgttagtctagtactgtaccccttcctatcttatccttcctttgcacccgactcaaaaaaaaagagtgcttcgagggtgcaaatccaactttctaagaaagttccctaaaccggggattcgccgagacaaacaagagacaaacggttttgaaagggggataggctatgcttttctttcattttttttttttttttttctgcctgctaaataaaaaaaaagggttggatatagccctctatcatatatatacaaatagaatagtccatttatacggactgctaagtgcggagacgggaatcgaacccgtgacctcaaggttatgagcctcgtgagctaccaaactgctctactccgctctgtagggccgaaaactggtggacgaaagaaaaaggtggaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctccgctttctagatgatccctatagaataggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaccttttttgtatcttcatccatagatcctttactcatatttattcaatcggaatacttatcggaatacttaatccaatgcaaaattatcgttcgcgactacgtactcataatcgaatttgtattttagatgcaaattcaattagtctttggatactaatcgggagaatgtatattcttcctcaatatgctattgagaggaaaaggattaaatcctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcggttggtacttcaatcgcggacctttactttaggatttagatagcccctctctagtctgtaaaatacatctcttcttaccataccaatagcgtatgaaccaaatgtatgcatttcgattaggatctattctacggttatgactacaaggatcattatttgtgaggacgtaaatgtgccagactgttgtaaggaatcgtttaattattcctacaatatatactaagagatataaaggcagtacagtccccataaatccctttcttccttttcttttttgttcagaattgaacaaagaaattgcgaaagatgttttcttcctccacgtatcatgaagtgcgagccatagggaaggagtgagatgactttccaaaatttttcatagacttcgtctatcgcttg

SEQ ID NO: 4 - DNA, 3411 bp, Zea mays
  source 1..3411: mol_type = genomic DNA
ctgcggaatcttctactggtacatggacaactgtttggactgatggacttaccagtcttgatcgttacaaaggacgatgctatcacatcgagcccgttcctggggacccagatcaatatatctgttatgtagcttatccattagacctatttgaagagggttctgttactaacatgtttacttccattgtgggtaacgtatttggtttcaaagccttacgcgctctacgtttggaggatctacgaattccccctgcttattcaaaaactttccaaggtccgcctcacggtatccaagttgaaagggataagttgaacaagtacggtcgtcctttattgggatgtactattaaaccaaaattgggattatccgcaaaaaattacggtagagcgtgttatgagtgtctacgcggtggacttgattttaccaaagatgatgaaaacgtaaactcacaaccatttatgcgctggagagaccgtttcgtcttttgtgccgaagcaatttataaagcacaagccgaaactggtgaaatcaaggggcattacttgaatgcgactgcaggtacatgcgaagaaatgattaagagagctgtatttgcaagggaattaggggttcctattgtaatgcatgactacttaacaggaggattcaccgcaaatactactttgtctcattattgccgcgacaacggcctacttcttcacattcaccgagcaatgcatgcagttattgatagacagaaaaatcatggtatgcatttccgtgtattagctaaagcattgcgtatgtcggggggagatcatatccactccggtacagtagtaggtaagttagaaggggaacgcgaaataactttaggttttgttgatttattgcgcgatgattttattgaaaaagatcgttctcgcggtatctttttcactcaggactgggtatccatgccaggtgttataccggtggcttctgggggtattcatgtttggcatatgccagctctgaccgaaatctttggagatgattccgtattacaatttggtggaggaactttaggacatccttggggaaatgcacctggtgcagcagctaatcgtgtggctttagaagcctgtgtacaagctcgtaacgaagggcgcgatcttgctcgtgaaggtaatgaaattatcaaagcagcttgcaaatggagtgctgaactagccgcagcttgtgaaatatggaaggagatcaaatttgatggtttcaaagcgatggataccatataaaataaaaaaaaaagcaaaatagaaagagaaaaaataagttaagaaatgcagtaattcttctttattcttctaattgattgcaattcaattcggctcaatcttttctaaaaaaaaaaaagactgagccgaattgaaatagatcttgatacgatcatgagacttgacaaatcgagattcttctattctatatatctagaatatagaaaggtataatacaataaacaaatataaatcaaaatatagtattatcgtacgataatggaatcaaatacgcagtatttacagaaaagagtcttcgtttattgggaaagaatcaatatacttttaatgtcgaatcgggattcactaagacagaaataaagcattgggtcgaactcttctttggtgttaaggtagtagctgtgaatagccatcgactacccggaaaggtagaagaatgggacctattctgggacatacaatgcattacagacgtatgatcattacccttcaactgggtattctattccacttctactttttaaattaaagggtattctgaaagaggtatttcttttattttcacaatagctttcttttgagcggccgcatccaatttcaagttcgattagaaggatagaaaggcgatgagaatgggaaaagaaaaataaaatattttattttaatgagttccccttttgagttccccttttttgcaattttcttattatcaattccattcatttattttatagaatacttttttattctatatctataaaaaatctttattttgtaaactaaaaaatacaatagtcaatattccttataatagatatacttaattatatcataagaatcttaagatatatttcgaatagatagaaatagtaaatttgaattgagacacatattctatgacagattttaacttaccctctattttcgtacctttagtaggcttagtatttccggcaattgcaatgacttccttatttctttatgtgcagaaaaataagattgtctagaaacgacggggccaaattttctcaatgtatttccaggatcataatacagatatttttttgtgtaagtactataatatgatagggtatgtagctctttctacacacaaatgaaaaacgtctatggatgcagatataagctacgagcataaatgcatgcatatgcgtagccgactatagcgagtttttttttttaagtggatccactaattttttttgaatagaaagtcaatgtatctaaccaattatttcacaggagtactagctactgaaggctatttcagaatcaaaaaaagtaaagtgaaaaatcatttagcttattctctcaatttcaattgaccgctactggatttagtatatctaatatgaattggcgatcagaacacatatggatagaacttctaaaaggttctcgaaaaagaggtaattttttctgggcctgtattctttttctaggttcattaggattcttagcggttggggcttccagttatcttggtaagaatatgatatctgtacttccatctcaacaaattcttttttttccacagggggtcgtgatgtctttctacggaatcgcgggcctattcattagctcctatttgtggtgcactattttatggaatgtaggcagtggttatgaccgattcgatagaaaagagggaatagtgtgcatttttcgttggggattccctggaataaaacgtcgcatcttccttcaattccttgtgcgggatatccaatcaattagaattcaggttaaagagggtctttatcctcgtcgtatcctttatatggaaatccggggccagggggtcattcccttgactcgtactgatgagaagttttttactccacgagaaattgaacaaaaagctgccgaattggcctatttcttgcgtgtaccaattgaagtattttgagtaccaattgcaattttttgcaattgtaaggggattttcgagtatttatctaaagtaaaggaaggaacaaacgaggataagagaaaattgcttcgaatttgttttgtccaagtgagatatatggcatactattcttccatttttatacgaaaggcctttttttatttctctattccactccatttagatctaagaaagaacccaatacaatgaaattccactagt

SEQ ID NO: 5 - DNA, 150 bp, Zea mays
  source 1..150: mol_type = genomic DNA
tctatgtattaatagaatctatagtattcttatagaataagaaaaaaaaaatgaagataataaactgcggattctttctttctcttccattcttacgtttccatattaaagtgtagtttttttacttaaatttaataatattaatctacc

SEQ ID NO: 6 - DNA, 173 bp, Zea mays
  source 1..173: mol_type = genomic DNA
ctcggatcctctatgtattaatagaatctatagtattcttatagaataagaaaaaaaaaatgaagataataaactgcggattctttctttctcttccattcttacgtttccatattaaagtgtagtttttttacttaaattttgtttaactttaagaaggagatatacatatg

SEQ ID NO: 7 - DNA, 230 bp, Zea mays
  source 1..230: mol_type = genomic DNA
actcccccaccacgatcgaacgggaatggataggaggcttgtgggattgacgtgatagggtagggttggctatactgctggtggcgaactccaggctaataatctgaagcgcatggatacaagttatccttggaaggaaagacaattccggatcctgtagaaataattttgtttaactttaagaaggagatatacccatgggtaaaggagaagaacttttcactggaatg

SEQ ID NO: 8 - DNA, 311 bp, Zea mays
  source 1..311: mol_type = genomic DNA
ctcactcccccaccacgatcgaacgggaatggataggaggcttgtgggattgacgtgatagggtagggttggctatactgctggtggcgaactccaggctaataatctgaagcgcttggatacaagttatccttggaaggaaagacaattccggatcctctatgtattaatagaatctatagtattcttatagaataagaaaaaaaaaatgaagataataaactgcggattctttctttctcttccattcttacgtttccatattaaagtgtagtttttttacttaaatttaataatattaatctaccatg

SEQ ID NO: 9 - DNA, 148 bp, Zea mays
  source 1..148: mol_type = genomic DNA
tagatttagggatttttcgagttgattcattccaccgcgaagtcccctgcatgggtatctaggaaatagttacttccaagtgaatcttccctagatacctaaaatctattttattatgatccatttcgcgaaaatatagattaagcta

SEQ ID NO: 10 - DNA, 150 bp, Nicotiana tabacum
  source 1..150: mol_type = genomic DNA
agaaattcaattaaggaaataaattaaggaaatacaaaaaggggggtagtcatttgtatataactttgtatgacttttctcttctatttttttgtatttcctccctttccttttctatttgtatttttttatcattgcttccattgaatt

SEQ ID NO: 11 - DNA, 201 bp, Nicotiana tabacum
  source 1..201: mol_type = genomic DNA
tgtctagagcgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgat

SEQ ID NO: 12 - DNA, 224 bp, Escherichia coli
  source 1..224: mol_type = genomic DNA
atcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaagct

SEQ ID NO: 13 - DNA, 792 bp, unidentified
  misc_feature 1..792: note = bacterial-derived aadA antibiotic resistance gene
  source 1..792: mol_type = genomic DNA
atggaagaagctgttattgctgaagtttctacacaattatctgaagttgttggagttattgaaagacatttagaacctacattattagctgttcatttatatggatctgctgttgatggaggattaaaacctcattctgatattgatttattagttactgttacagttagattagatgaaactacaagaagagctttaattaatgatttattagaaacttctgcttctcctggagaatctgaaattttaagagctgttgaagttactattgttgttcatgatgatattattccttggagatatcctgctaaaagagaattacaatttggagaatggcaaagaaatgatattttagctggaatttttgaacctgctactattgatattgatttagctattttattaacaaaagctagagaacattctgttgctttagttggacctgctgctgaagaattatttgatcctgttttagaacaagatttatttgaagcattaaatgaaacattaactttatggaactctcctcctgattgggctggagatgaaagaaatgttgttttaacattatccagaatttggtattctgctgttacaggaaaaattgctcctaaagatgttgctgctgattgggctatggaaagattacctgctcaatatcaacctgttattttagaagctagacaggcttatttaggacaagaagaagatagattagcttcaagagctgatcaattagaagaatttgttcattatgttaaaggagaaattacaaaagttgttggaaaataa

SEQ ID NO: 14 - DNA, 798 bp, unidentified
  misc_feature 1..798: note = nptII antibiotic resistance marker
  source 1..798: mol_type = genomic DNA
atggggattgaacaagatggattgcacgcaggttctccggccgcttgggtggagaggctattcggctatgactgggcacaacagacaatcggctgctctgatgccgccgtgtttcggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctag

SEQ ID NO: 15 - DNA, 780 bp, unidentified
  misc_feature 1..780: note = Antibiotic resistance marker aphA-6
  source 1..780: mol_type = genomic DNA
atggaattaccaaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaattcatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagatcttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattga

SEQ ID NO: 16 - DNA, 20 bp, Synthetic construct
  misc_feature 1..20: note = Forward primer
  source 1..20: mol_type = other DNA
gacatcacgtccctgctcaa

SEQ ID NO: 17 - DNA, 20 bp, Synthetic construct
  misc_feature 1..20: note = Reverse primer
  source 1..20: mol_type = other DNA
ttcccaacattcccataggc

SEQ ID NO: 18 - DNA, 21 bp, Synthetic construct
  misc_feature 1..21: note = Primer
  source 1..21: mol_type = other DNA
gcctccatttcgtcgaatccc

SEQ ID NO: 19 - protein, 326 aa, Zea mays
MACSLRELRTRIDSVRNTQKITEAMKLVAAAKVRRAQEAVVSSRPFSEALVEVLYNMNQEIQTEDIDLPLTRTRPVKKVALVVLTGERGLCGSFNNNVLKKAETRIDELKQLGLQYTVVSVGKKGNAYFQRRPYIPLERDLEVSSVPTVKDSQAICDLVYSLFVSEEVDKVELLYSKFVSLVRSDPIIQTLLPMSPKGEICDVNGVCVDATEDELFRLTTKEGKLTVEREKVKIETQPFSPVVQFEQDPVQILDALLPLYLNSQILRALQESLASELAARMSAMSSATDNAIELRKNLSIAYNRQRQAKITGEILEIVAGADALAG

SEQ ID NO: 20 - DNA, 978 bp, Zea mays
  source 1..978: mol_type = genomic DNA
atggcttgttctttacgtgaattacgtactcgtattgattctgttcgaaatactcaaaaaattactgaagctatgaaattagttgctgctgctaaagtacgtcgtgctcaagaagcagtagtttcttctcgacctttttccgaagcattagttgaagtactttataatatgaatcaagaaattcaaacagaagatattgatttacctttaactcgtactcgtcctgttaaaaaagttgctttagtagttcttacaggagaacgaggattatgtggttcctttaataataatgtattaaaaaaagctgaaactcgtattgatgaattaaaacaacttggattacaatatacagtagtttccgttggaaaaaaaggaaatgcttattttcaacgtcgtccttatattcctcttgaacgtgatttagaggtatcctccgttcctactgtaaaagattcccaagctatttgtgatttagtttattctctttttgtttccgaagaagtagataaagttgaacttttatattccaaatttgtatctttagttcgttctgatcctattattcaaactcttcttcctatgtcccctaaaggagaaatatgtgatgtaaatggagtttgtgtagatgctactgaagatgaactttttcgtcttactactaaagagggaaaactcacagtagaacgagaaaaagtaaaaattgaaacacaacctttttcccctgttgtacaatttgagcaagatcctgttcaaattttagatgctcttcttcctttatatttaaattctcaaatattacgtgctcttcaagaatctcttgcttctgaattagctgctcgtatgtccgcaatgtcttccgctactgataatgcaattgaattacgaaagaatctttccattgcttataatcgacaacgtcaagctaaaataactggagaaattttagagatagtagctggagcagatgctttagctgga

SEQ ID NO: 21 - protein, 350 aa, Zea mays
MASAAKETQRPVYPFAAIVGQDEMKLCLLLNVIDPKIGGVMIMGDRGTGKSTTVRSLVDLLPDIRVVVGDPFNSDPDDPEVMGPEVRQRVLQGDTGLPVTTAKITMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDRDPKTFRESYHDEQEKLQQQISSARSNLGAVQIDHDLRVKISKVCSELNVDGLRGDIVTNRAAKALAALKGRDSVTVEDIATVIPNCLRHRLRKDPLESIDSGLLVIEKFYEVFS

SEQ ID NO: 22 - DNA, 1053 bp, Zea mays
  source 1..1053: mol_type = genomic DNA
atggcttctgctgctaaagaaactcaaagacctgtttatccttttgcagcaattgtaggacaagatgaaatgaaactttgtcttttattaaatgtaattgatcctaaaattggaggagttatgattatgggtgatagaggtactggaaaatctactacagttcgctctttagttgatttacttcctgatattagagttgttgttggagatccttttaattctgatcctgatgatccagaagtaatgggaccagaagtaagacagagagttttacaaggagatactggattacctgttactactgctaaaattacaatggtagatttacctttaggagctacagaagatagagtatgtggaactattgatattgaaaaagctcttactgaaggagttaaagcatttgaacctggtcttcttgctaaagctaatagaggtattttatatgttgatgaagttaatttattagatgatcacttagttgatgtacttcttgattctgctgcttctggatggaatactgtagaaagagaaggtatttccatttcccatccagctagatttatattaataggatctggaaatcctgaagaaggagaacttcgtcctcaattattagatagatttggtatgcatgcacaagttggtacagttagagatgcagaattacgtgttaaaattgttgaagaaagagctagatttgatcgagatccaaaaacttttcgagaatcttatcatgatgaacaagaaaaattacaacaacaaatatcttctgcacgaagtaatcttggagctgtacaaattgatcatgatcttagagtaaaaataagtaaagtttgctctgaattgaatgttgatggacttagaggagatattgttactaatagagctgctaaagcacttgctgcattaaaaggaagagattctgttactgttgaagatattgctactgtaattcctaattgtttaagacatcgtttacgtaaagatcctcttgaatctattgattctggattacttgttattgaaaaattttatgaagttttttcttag

SEQ ID NO: 23 - DNA, 20 bp, Synthetic construct
  misc_feature 1..20: note = Forward primer
  source 1..20: mol_type = other DNA
ttcgggacaggcttgaaact

SEQ ID NO: 24 - DNA, 20 bp, Synthetic construct
  misc_feature 1..20: note = Reverse primer
  source 1..20: mol_type = other DNA
ccgtctggatctcctggttc

SEQ ID NO: 25 - DNA, 19 bp, Synthetic construct
  misc_feature 1..19: note = Forward Primer
  source 1..19: mol_type = other DNA
aggccaacaggggcatact

SEQ ID NO: 26 - DNA, 20 bp, Synthetic construct
  misc_feature 1..20: note = Reverse primer
  source 1..20: mol_type = other DNA
cgctgtcccttcctttcaac

SEQ ID NO: 27 - protein, 786 aa, Zea mays
MEATGRGLFPNKPTLPAGPRKRGPLLPAAPPPPSPSSLPLDSLLLHLTAPAPAPAPAPRRSHQTPTPPHSFLSPDAQVLVLAISSHPLPTLAAFLASRRDELLRADITSLLKALELSGHWEWALALLRWAGKEGAADASALEMVVRALGREGQHDAVCALLDETPLPPGSRLDVRAYTTVLHALSRAGRYERALELFAELRRQGVAPTLVTYNVVLDVYGRMGRSWPRIVALLDEMRAAGVEPDGFTASTVIAACCRDGLVDEAVAFFEDLKARGHAPCVVTYNALLQVFGKAGNYTEALRVLGEMEQNGCQPDAVTYNELAGTYARAGFFEEAARCLDTMASKGLLPNAFTYNTVMTAYGNVGKVDEALALFDQMKKTGFVPNVNTYNLVLGMLGKKSRFTVMLEMLGEMSRSGCTPNRVTWNTMLAVCGKRGMEDYVTRVLEGMRSCGVELSRDTYNTLIAAYGRCGSRTNAFKMYNEMTSAGFTPCITTYNALLNVLSRQGDWSTAQSIVSKMRTKGFKPNEQSYSLLLQCYAKGGNVAGIAAIENEVYGSGAVFPSWVILRTLVIANFKCRRLDGMETAFQEVKARGYNPDLVIFNSMLSIYAKNGMYSKATEVFDSIKRSGLSPDLITYNSLMDMYAKCSESWEAEKILNQLKCSQTMKPDVVSYNTVINGFCKQGLVKEAQRVLSEMVADGMAPCAVTYHTLVGGYSSLEMFSEAREVIGYMVQHGLKPMELTYRRVVESYCRAKRFEEARGFLSEVSETDLDFDKKALEAYIEDAQFGR

SEQ ID NO: 28 - DNA, 2673 bp, Zea mays
  source 1..2673: mol_type = genomic DNA
ctccccgtccacccgctcccgccaccgtatccgtaaacccaatcccccatttcccctagggggcatgctgcgcgcgggcctcccctccacgctcaccagctcgtagccgtttcctgccgctcctggctcctgcaccgagcggcaaacccgcgccgccgccgccgccgatggaggccaccggcagggggctgttcccgaacaagcccaccctcccggcggggccgaggaaacggggcccgctcctcccggccgcgcccccgccaccgtccccctcctcgctcccgctcgactcgctcctgctccacctcaccgcgcccgcccccgcgccggcccccgcgccgcggcggtcgcaccagacgccgacgccgccgcactccttcctctcccccgacgcgcaggtgctggtgctcgccatctcctcgcacccgctccccacgctggcggccttcctggcctcccgccgcgacgagctcctccgcgcggacatcacgtccctgctcaaggcgctggagctctcggggcactgggagtgggcgctcgcgctcctccggtgggcaggcaaggagggtgccgccgacgcgtcggcgctcgagatggtcgtccgcgcgctgggccgcgagggccagcacgacgccgtctgcgcgctgctcgacgaaacgccgctcccgccgggctcccgcctcgacgtccgcgcctacaccaccgtgctgcacgcgctctcccgcgcgggccggtacgagcgcgcgctcgagctcttcgccgagctccggcgccagggggtggcgcccacgctcgtcacctacaacgtcgtgctggacgtgtacgggcggatgggccggtcgtggccgcggatcgtcgccctcctcgatgagatgcgcgccgccggggtcgagcccgacggcttcaccgccagcacggtgatcgccgcgtgctgccgcgacgggctggttgacgaggcggtggcgttcttcgaggacctcaaggcccgcggccacgccccgtgcgtcgtcacgtacaacgcgttgctccaggtgttcggcaaggccgggaactacacggaggcgctgcgcgtgctcggggagatggagcagaacggctgccagccagatgctgtgacgtacaacgagctcgccggaacgtacgcccgggctgggttcttcgaggaggctgccaggtgcctggacacaatggcatccaagggtctgttgccaaacgcattcacgtacaacaccgtgatgacagcctatgggaatgttgggaaggtggatgaggcgctcgctctgtttgaccagatgaagaagaccgggttcgtgccgaacgtgaacacgtacaatcttgtccttggcatgcttggcaagaagtcaaggttcacggtgatgctagagatgcttggagagatgtcgaggagcggatgcacaccgaaccgggtaacatggaacacaatgcttgcagtctgtgggaagcgtggcatggaggactacgtcacccgggttctggaggggatgaggtcttgcggggttgaactgagccgagacacctacaacaccctgatagctgcgtacggccggtgtggctcgaggactaatgccttcaagatgtacaacgagatgaccagcgctggattcaccccctgcatcaccacgtacaacgcgttgctgaacgtgctgtcgcggcagggcgactggtccaccgcccagtcgatcgtaagcaaaatgaggaccaaggggttcaagccgaacgagcagtcgtattcgctgctgctccagtgctacgcgaaggggggcaacgtggcagggatagccgcgatcgagaacgaggtgtacggatcaggtgccgttttcccaagctgggtgatcctgaggacccttgtcatcgccaatttcaagtgccggcgactggatggcatggagacggcgtttcaagaggtgaaggccagaggctacaacccggacctcgtgatattcaactccatgctgtccatctacgcgaagaacgggatgtacagcaaggccaccgaggtcttcgactccatcaagcggagcgggctgagccccgacctcatcacctacaacagcctgatggacatgtacgccaagtgcagcgagtcgtgggaggccgagaagatactgaaccagctcaagtgctcccagacgatgaagcccgacgtggtgtcctacaacacggtcataaacgggttctgcaagcaggggctggtgaaagaggcccagagggtcctctcggagatggtcgccgacggcatggccccctgcgccgtgacctaccacacgctcgtcgggggttactccagcctggagatgttcagcgaggccagggaggtcatcggctacatggtccagcacggcctcaagcctatggagctgacctacaggagagtcgtcgagagctactgcagagcgaagcggttcgaggaggctcgcggcttcctgtccgaggtctcggagaccgacctggattttgacaagaaggcgctcgaagcctatatagaggatgcgcagtttggaaggtagagagagggagagtggctgtgctaagaggtgggcttaggtacagttcgtacggttttctttggttacaggcaactgtcggtttgcaattcgcttgttatatatatatgctcaagttttgataaaaaaaaaaaaaaaaaaaaaaaaa

SEQ ID NO: 29 - DNA, 1323 bp, Zea mays
  source 1..1323: mol_type = genomic DNA
tcttccgcttccccccgaccccgcgcgccgcggaccagcagccatgtcgtgctcccacctctccaccgcctggtcctcctcggcgctcgccagcagcgcctccaccacccgacggcgctcccccccgcgctcgggtctcctggtgcggtgttccctccgtgagctccgcacccgcatcgactccgtcaggaacacgcagaagatcacggaggccatgaagctggtggccgccgccaaggtccggcgcgcgcaggaggccgtcgtctcctctcgccctttctcggaggcgctggtggaggtgctctacaacatgaaccaggagatccagacggaggacatcgacctgcccctcacccgcacccgccccgtcaagaaggtggccctcgtggtcctgaccggcgagcgcggcctctgcgggagcttcaacaacaacgtgctcaagaaggcggaaacccgcatcgatgagctcaagcagctgggcctccagtacaccgtcgtcagcgtggggaagaagggcaacgcctacttccagcgccgcccctacatcccgttggagcgcgatctagaggtgagcagcgtgcccaccgtcaaggactcgcaggccatctgcgacctcgtctactcgctcttcgtctccgaggaggtggacaaggtggagctgctctactccaagttcgtgtcgctggtgcgctccgaccccatcatccagacgctgctccccatgtcgcccaagggcgagatctgcgacgtcaacggcgtctgcgtggacgccaccgaggacgagctcttccgcctcaccaccaaggaggggaagctcaccgtggagcgcgagaaggtgaagatcgagacgcagcccttctcccccgtggtgcagttcgagcaggaccccgtgcagatcctggacgcgctgctcccgctctacctcaacagccagatcctccgtgccctgcaggagtcgctcgccagcgagctcgccgcccggatgagcgccatgagcagcgccaccgacaacgccatcgagctccgcaagaacctctccatcgcctacaaccgacagcgccaggccaagatcaccggggagatcctcgagatcgtcgccggtgccgacgccctcgctggctgagcctgagtccccttggtggtgcggtgctcctgctatatataattctttttcctcattttctccactctgcctgtaattcctgataatagcacaaacgcaacggagttctgtgttccttgcgacacacaagaatttcaatccagagatgccaaaatgtataatcttgccgcaactgagacgtcgaagctcagtgtattttt

SEQ ID NO: 30 - protein, 359 aa, Zea mays
MSCSHLSTAWSSSALASSASTTRRRSPPRSGLLVRCSLRELRTRIDSVRNTQKITEAMKLVAAAKVRRAQEAVVSSRPFSEALVEVLYNMNQEIQTEDIDLPLTRTRPVKKVALVVLTGERGLCGSFNNNVLKKAETRIDELKQLGLQYTVVSVGKKGNAYFQRRPYIPLERDLEVSSVPTVKDSQAICDLVYSLFVSEEVDKVELLYSKFVSLVRSDPIIQTLLPMSPKGEICDVNGVCVDATEDELFRLTTKEGKLTVEREKVKIETQPFSPVVQFEQDPVQILDALLPLYLNSQILRALQESLASELAARMSAMSSATDNAIELRKNLSIAYNRQRQAKITGEILEIVAGADALAG

SEQ ID NO: 31 - DNA, 1323 bp, Zea mays; identical to SEQ ID NO: 29
  source 1..1323: mol_type = genomic DNA

SEQ ID NO: 32 - DNA, 1691 bp, Zea mays
  source 1..1691: mol_type = genomic DNA
gcctccaatcgcagcatccctccgagatcgctgccccgtccccctcccccggcttatcccctatcccctcctcccccgctccttccacacaaacctccatgccccttcttcctctcctccccgtcatggcttccaccttctcccccacttccgccgcgagggccctcctcccgggctccacctcccgcccactcttcctcgccgcttcagcttcctcaggtaaccgtcatctcacatttccctccccctactcactctgtttcccagctcgctcatcagcgagcgccgaatccaacacccccccttaccgcccccccggtacactcgcagggcgcattcaaccatccaggaagggactggacttccgccgcggccgattcaccgtctgcaatgtcgccgctcccaccgccgccgaacaggtacgcccccgttctccaatcccgcagcccgcaggaaccggtcggtaatccttcctccgcaaccccgcattttcgatgcctgcttctgcttccgcgcaggaggcgacggcgtcggccgcgaaggagacccagcgccccgtgtacccgttcgcggccatcgtggggcaggacgagatgaagctctgcctgctgctcaacgtcatcgaccccaagatcggcggcgtcatgatcatgggcgacaggggcacggggaagtccaccaccgtccgctccctcgtcgacctgctcccggacatccgcgtcgtcgtcggcgaccccttcaactccgacccggacgaccccgaggtcatgggccccgaggtccgccagcgggtcctgcagggggacaccggcctccccgtcaccaccgccaagatcaccatggtcgacctgcccctcggcgccaccgaggaccgcgtctgcggcaccattgacatcgagaaggcgctcaccgagggcgtcaaggcgttcgagcccggcctgctcgccaaggccaacaggggcatactgtacgtcgacgaggtcaacctgctggacgaccacctcgtcgacgtgctgctggattccgctgcgtcggggtggaacacggtggagagggagggtatctccatatcccaccctgctcgcttcatcctcatcggctctggtaacccggaggaaggggagctcaggccccagctgctggaccggttcgggatgcacgcgcaggttggtaccgtcagggacgccgagctcagggtgaagatcgtggaggagagggctcgtttcgacagggatccgaagacgttccgtgagtcgtatcatgacgagcaggagaagctccagcagcagatatcatctgcacggagtaaccttggcgctgtgcagattgaccatgacctccgtgtcaagatatccaaggtgtgctctgagttgaacgttgatggactcagaggtgacattgtgactaacagggctgccaaggcgctggctgcgttgaaaggaagggacagcgtcaccgtggaggacattgctactgtcattccaaactgcttgaggcatcggctccgcaaggatccgcttgaatccattgactcgggtttacttgtcattgagaagttttatgaagtctttagctagattgttcttgaggtaaatgttccttagtcacaatttttggcgggaaccctcttgttctgttactttcataatgttctgctgtttaataatatctggagcttgaattggtatcaa

SEQ ID NO: 33 - protein, 422 aa, Zea mays
MPLLPLLPVMASTFSPTSAARALLPGSTSRPLFLAASASSGRIQPSRKGLDFRRGRFTVCNVAAPTAAEQEATASAAKETQRPVYPFAAIVGQDEMKLCLLLNVIDPKIGGVMIMGDRGTGKSTTVRSLVDLLPDIRVVVGDPFNSDPDDPEVMGPEVRQRVLQGDTGLPVTTAKITMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDRDPKTFRESYHDEQEKLQQQISSARSNLGAVQIDHDLRVKISKVCSELNVDGLRGDIVTNRAAKALAALKGRDSVTVEDIATVIPNCLRHRLRKDPLESIDSGLLVIEKFYEVFS

SEQ ID NO: 34 - DNA, 1686 bp, Zea mays
  source 1..1686: mol_type = genomic DNA
actagatagatgctgtcatgctgaggacgaaggcaagcatgttgggcacagcgccatagtatcaaactatcattttgagccgtttcgcgtaccctaaaatcagtggcaacagcgccatagcatcgtcaactccatcaagccgttaccatcaacaacgattgcttgctgccgactgccaagcggggccggtcaatccagaagttggcagcctccaatcgcagcatccctccgagatcgctgccccgtccccctcccccggcttatcccctatcccctcctcccccgctccttccacacaaacctccatgccccttcttcctctcctccccgtcatggcttccaccttctcccccacttccgccgcgagggccctcctcccgggctccacctcccgcccactcttcctcgccgcttcagcttcctcagggcgcattcaaccatccaggaagggactggacttccgccgcggccgattcaccgtctgcaatgtcgccgctcccaccgccgccgaacaggaggcgacggcgtcggccgcgaaggagacccagcgccccgtgtacccgttcgcggccatcgtggggcaggacgagatgaagctctgcctgctgctcaacgtcatcgaccccaagatcggcggcgtcatgatcatgggcgacaggggcacggggaagtccaccaccgtccgctccctcgtcgacctgctcccggacatccgcgtcgtcgtcggcgaccccttcaactccgacccggacgaccccgaggtcatgggccccgaggtccgccagcgggtcctgcagggggacaccggcctccccgtcaccaccgccaagatcaccatggtcgacctgcccctcggcgccaccgaggaccgcgtctgcggcaccattgacatcgagaaggcgcttaccgagggcgtcaaggcgttcgagcccggcctgctcgccaaggccaacaggggcatactgtacgtcgacgaggtcaacctgctggacgaccacctcgtcgacgtgctgctggattccgctgcgtcggggtggaacacggtggagagggagggtatctccatatcccaccctgctcgcttcatcctcatcggctctggtaacccggaggaaggggagctcaggccccagctgctggaccggttcgggatgcacgcgcaggttggtaccgtcagggacgccgagctcagggtgaagatcgtggaggagagggctcgtttcgacagggatccgaagacgttccgtgagtcgtatcatgacgagcaggagaagctccagcagcagatatcatctgcacggagtaaccttggcgctgtgcagattgaccatgacctccgtgtcaagatatccaaggtgtgctctgagttgaacgttgatggactcagaggtgacattgtgactaacagggctgccaaggcgctggctgcgttgaaaggaagggacagcgtcaccgtggaggacattgctactgtcattccaaactgcttgaggcatcggctccgcaaggatccgcttgaatccattgactcgggtttacttgtcattgagaagttttatgaagtctttagctagattgttcttgaggtaaatgttccttagtcacaatttttggcgggaaccctcttgttctgttactttcataatgttctgctgtttaataatatctggagcttgaattggtatc

SEQ ID NO: 35 - DNA, 1108 bp, Glycine max
  source 1..1108: mol_type = genomic DNA
gagggagagaatctaacaggactgggaggatagaactgaagtggctgtgtggtgttacagttgagttgagcgatctcttctcttttcttgtcttgtcttttgcaatggctaatgcaattataatgccgggttttctgcttcgacccactacccagacgacgccgtttcggttgcccaatcacactcactccttccattcccctcgctgctccctttccctcacaccccaatcggctaaagcttttgcagttccaaggaggaaagcattgtccttgatcttatcgagttactttctctcggaggctggtttagcattggctcaacagtctcctgtgttccgcgaatacatagacgcatttgatggttattcattccagtacccggggagttggattcaagtccgaggtgccggggccgacatattcttcagggacccttttgttcttgatgaaaatctatctcttgaaatttcttcaccttcatcttcccagtacaagagtgttgaagacttgggtccaccccaagaagctggaaagaaagtgctcaaacagtatctgactgagtttatgtctactagactcggcgttagacgtgaatcaaatattctttccacgtcttcaagagtagctgatgatggcaggttatactatcaagttgaggtaaacataaagtcatatgccaataacaacgagcttgctgttatgccagaagcccgagtggtacgtctggaatgggatcggaggtacctatctgttcttggggttgaaaacaaccaattgtatgagttgagattgcaggtgccagaaaatgtgttttcagaggaggaaaatgatcttcgtcgaatcatggattcgtttcgagtgaacaagattgccgcttagaactagagtgttggatacatgaagatattccacggtctttgaattttaatcttatttcttctttattctgatgatctctttctctaatctttttcttttttacttgattgaatttttttcttctttggctaaattttggccatactatcatatcatttgttacattattgttctcactttcctatttatgtgagtctgcttgttaaattaattttaacatgactgtt

SEQ ID NO: 36 - DNA, 2450 bp, Glycine max
  source 1..2450: mol_type = genomic DNA
gggaggccaataggaagcacgccagctgctacgaatggtgaataaaagacaaaagaaacaaactgctacatagcatacagtctgtcttctcttctcttctccggttatggcgtccgccttgggcacttcttcaattgcggttctgccttcgcgctacttctcttcttcttcttccaagccttccattcacactctctctctaacttcaggtacattgcttgcaaagtctatgttttttgtctgttttttgcctctaatatcttgttttggatggtgtgctgcaaacccatttcatatattttgcttctctgtgctaaagttgtgattttattttgcctggggggttgtagctggaaatggaaggtgggtctttgtgggcattatgtgatagtttgaacttagaatcttagttgagtggttgtgaatttgcagggcagaactatgggcggaagttttatggaggaattggaatccatggcataaagggaagggctcagctctcggttaccaatgttgccactgaagttaactctgtagaacaggtaggtaggatattggatttttgaggtctagatgttctttcagtgtctttgcctgtgttttgaattgcattgggtggatatactaggtgcacccgagattaattgttgtaaagatttccattcttttgtgtgttgtgtgtgttgagtcattgagcattcattcctagtgaattttaaacaaattatgggcagaagacaatttttagactatattgtgaatttgtgatacttttcttgggtttaatcaacatgcacggtaatataaaggctttacattatcaattaatccaaaatcattttttgtatgacctttaagataactgttgtaaaatttaacaaacttaccgtgcatgactgcagttcatgattggataatagtataaaagtataaaacacttttacattgttagtgcatatattatttattcatttttcttttccctttaaacgtagatggtttctgttaccattcacatgcacatttagagtgtggttgattggcggaaaatagcaaggcgaaaaatagaggaatgtaaatgaggcagtatgtgaacttatatttttgtcatgcattttttccgttcaaaattagtgctagatttcttagaaattaaggattgggggccaggcctttgcattttgattctttgtgtggttgtgtgagtgttacaataatctaaccctgtaaacttttcgtatccaggctcagagtattgcttctaaagaaagccagaggccagtatacccattttctgccatagttggacaagatgagatgaagctttgtcttctccttaatgtgattgatcctaagattggaggtgtaatgatcatgggggataggggcacagggaaatctacaacggtcaggtcattggttgatttacttcccgaaatcaaggttgttgctggtgacccttataactcagacccacaagatccagaattcatgggtgttgaagtcagagagcgtgtacttcaaggagaggaactttctgttgtcttgaccaaaattaacatggttgatttgccattgggagctacagaagatagagtgtgtggaacgattgacattgagaaagccctgactgagggtgtcaaggcatttgagcctggactactggctaaagctaataggggaatcttatatgttgatgaagttaatcttttggatgatcacttggtggatgtgttgttggattctgctgcatcaggatggaacacagtagagagagagggaatttctatctcgcatcctgcacggtttatcctaattggctcaggcaaccccgaagaaggggagctccggccgcagctgctagataggtttggaatgcatgctcaagttggaactgttagggatgctgagctcagagtgaagattgtggaggagagaggtcgatttgacaaaaatccaaaggaatttcgagattcttacaaagctgagcaagagaagctccaacaacaaattacctcagcaaggagtgttctttcttctgttcaaattgatcaagatctcaaggtgaaaatctccaaggtgtgtgctgagttgaatgtggatggattaagaggagacatagtaacaaatagagctgcaaaagctctggctgctctgaagggaagagacaacgtaagtgccgaggatattgctactgtcatccctaactgcttgagacaccgtcttagaaaggatcccttggagtctatagactcaggtttacttgtcactgagaaattttatgaggtattcagctgaggatcatagatgtaacacggtttgtattctgtggtattccatgagtataatgctacttcattatttacattttaccttttctgtggagattttcattgtcgttaaacatcaataagattcatgagttccttctacacaggaaattccgagca

SEQ ID NO: 37 - DNA, 1539 bp, Glycine max
  source 1..1539: mol_type = genomic DNA
gggaggccaataggaagcacgccagctgctacgaatggtgaataaaagacaaaagaaacaaactgctacatagcatacagtctgtcttctcttctcttctccggttatggcgtccgccttgggcacttcttcaattgcggttctgccttcgcgctacttctcttcttcttcttccaagccttccattcacactctctctctaacttcagggcagaactatgggcggaagttttatggaggaattggaatccatggcataaagggaagggctcagctctcggttaccaatgttgccactgaagttaactctgtagaacaggctcagagtattgcttctaaagaaagccagaggccagtatacccattttctgccatagttggacaagatgagatgaagctttgtcttctccttaatgtgattgatcctaagattggaggtgtaatgatcatgggggataggggcacagggaaatctacaacggtcaggtcattggttgatttacttcccgaaatcaaggttgttgctggtgacccttataactcagacccacaagatccagaattcatgggtgttgaagtcagagagcgtgtacttcaaggagaggaactttctgttgtcttgaccaaaattaacatggttgatttgccattgggagctacagaagatagagtgtgtggaacgattgacattgagaaagccctgactgagggtgtcaaggcatttgagcctggactactggctaaagctaataggggaatcttatatgttgatgaagttaatcttttggatgatcacttggtggatgtgttgttggattctgctgcatcaggatggaacacagtagagagagagggaatttctatctcgcatcctgcacggtttatcctaattggctcaggcaaccccgaagaaggggagctccggccgcagctgctagataggtttggaatgcatgctcaagttggaactgttagggatgctgagctcagagtgaagattgtggaggagagaggtcgatttgacaaaaatccaaaggaatttcgagattcttacaaagctgagcaagagaagctccaacaacaaattacctcagcaaggagtgttctttcttctgttcaaattgatcaagatctcaaggtgaaaatctccaaggtgtgtgctgagttgaatgtggatggattaagaggagacatagtaacaaatagagctgcaaaagctctggctgctctgaagggaagagacaacgtaagtgccgaggatattgctactgtcatccctaactgcttgagacaccgtcttagaaaggatcccttggagtctatagactcaggtttacttgtcactgagaaattttatgaggtattcagctgaggatcatagatgtaacacggtttgtattctgtggtattccatgagtataatgctacttcattatttacattttaccttttctgtggagattttcattgtcgttaaacatcaataagattcatgagttccttctacacaggaaattccgagcaaaaaaaaaaaaaaaa

SEQ ID NO: 38 - protein, 421 aa, Glycine max
MASALGTSSIAVLPSRYFSSSSSKPSIHTLSLTSGQNYGRKFYGGIGIHGIKGRAQLSVTNVATEVNSVEQAQSIASKESQRPVYPFSAIVGQDEMKLCLLLNVIDPKIGGVMIMGDRGTGKSTTVRSLVDLLPEIKVVAGDPYNSDPQDPEFMGVEVRERVLQGEELSVVLTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERGRFDKNPKEFRDSYKAEQEKLQQQITSARSVLSSVQIDQDLKVKISKVCAELNVDGLRGDIVTNRAAKALAALKGRDNVSAEDIATVIPNCLRHRLRKDPLESIDSGLLVTEKFYEVFS

SEQ ID NO: 39 - DNA, 1266 bp, Glycine max
  source 1..1266: mol_type = genomic DNA
atggcgtccacgttgggcacttcttcaattgcggttcttccttcgcgctgcatctcttctttttcttccaagccttccattcacacactctctctaacttcagggcagagctatgggcggaaattttatggaggaattggaattcatggcatcaagggaaggtctcagctctcagttgccaatgttgccactgaagttaactctgtagaacaggcccaaagtattgcttctaaagaaagccagaggccagtatacccattttctgccatagttggacaagatgagatgaagctttgtcttctccttaatgtgattgatcctaagattggaggtgtaatgatcatgggggataggggcacagggaaatctacaaccgtcaggtcattggttgatttacttcctgaaatcaaggttgttgctggtgacccttataactcagacccacaagatccagaattcatgggtgttgaagtcagagagcgtgtacttcaaggagaggaactttctgttgtcttgaccaaaattaacatggttgatttgccattgggagctacagaagatagagtgtgtggaacaattgacattgagaaagccctgactgagggtgtcaaggcatttgagcctggactactggctaaagctaataggggaatcctatatgttgatgaagttaatcttttggatgatcacttggtagatgtgttgttggattctgctgcatcaggatggaacacagtggagagagagggaatttctatctcacaccctgcacggtttatcctaattggctcgggaaaccctgaagaaggtgagctccggccacagctgctggataggtttggaatgcatgctcaagtgggaactgttagggatgctgagcttagagtgaagattgtggaggagagaggtcgatttgacaaaaatccaaaggaattccgagattcttacaaagccgagcaagagaagctccaacaacaaattacatcagcaaggagtgttctttcttctgttcagattgatcaagatctcaaggtgaaaatctccaaggtgtgtgctgagttgaatgtggatggattaagaggagacatagtaacaaatagagctgcaaaagctcttgctgctctgaaggaaagagacaaagtaagtgcagaggatattgctactgtcatccctaactgcttgagacaccgtcttagaaaggatcccttggagtctatagactcaggtttacttgtcactgagaaattttatgaggtatttagctga

SEQ ID NO: 40 - protein, 421 aa, Glycine max
MASTLGTSSIAVLPSRCISSFSSKPSIHTLSLTSGQSYGRKFYGGIGIHGIKGRSQLSVANVATEVNSVEQAQSIASKESQRPVYPFSAIVGQDEMKLCLLLNVIDPKIGGVMIMGDRGTGKSTTVRSLVDLLPEIKVVAGDPYNSDPQDPEFMGVEVRERVLQGEELSVVLTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERGRFDKNPKEFRDSYKAEQEKLQQQITSARSVLSSVQIDQDLKVKISKVCAELNVDGLRGDIVTNRAAKALAALKERDKVSAEDIATVIPNCLRHRLRKDPLESIDSGLLVTEKFYEVFS

(19) United States
(12) Patent Application Publication
(10) Pub. No.: US 2023/0313212 A1
(43) Pub. Date: Oct. 5, 2023

(54) PLASTID TRANSFORMATION BY COMPLEMENTATION OF NUCLEAR MUTATIONS

(71) Applicant: PLASTOMICS INC., St. Louis, MO (US)

(72) Inventor: Jeffrey Staub, Wildwood, MO (US)

(21) Appl. No.: 18/180,488

(22) Filed: Mar. 8, 2023

Related U.S. Application Data
(63) Continuation of application No. PCT/US2021/048408, filed on Aug. 31, 2021.
(60) Provisional application No. 63/180,783, filed on Apr. 28, 2021.
(60) Provisional application No. 62/706,758, filed on Sep. 9, 2020.

(51) Int. Cl. C12N 15/82

(52) U.S. Cl. CPC C12N 15/8214; C12N 15/8269

(57) ABSTRACT

The disclosure provides a method of expressing an agronomically beneficial trait in a plant plastid comprising expressing an exogenous nucleic acid in the plant to produce non-photosynthetic mutant plants, and using callus grown from the mutant plants as recipients for introduction of a construct having a functional copy of the mutated gene and a gene conferring an agronomically beneficial trait. Embodiments provide for mutations in chloroplast-encoded genes, as well as mutations in nuclear-encoded genes targeted to the chloroplast that are required for photosynthesis. The disclosure also provides plants and plant parts produced from such methods, as well as kits for performing the methods as described.

### CROSS-REFERENCE TO RELATED APPLICATIONS

This application is a bypass continuation of International Application No. PCT/US2021/048408 filed on Aug. 31, 2021, and published as WO 2022/055751A1 on Mar. 17, 2022 and which claims priority to and the benefit of U.S. Provisional Application Ser. No. 62/706,758 filed on Sep. 9, 2020 and Ser. No. 63/180,783 filed on Apr. 28, 2021 the contents of which are herein incorporated in their entireties.

### INCORPORATION OF SEQUENCE LISTING

The sequence listing that is contained in the file named “2023-03-08_Sequence_Listing_P1810002US1.xml” which is 72 kilobytes as measured in Microsoft Windows operating system and was created on Sep. 10, 2019, and updated on Mar. 8, 2023 is filed electronically herewith and incorporated herein by reference.

### FIELD OF THE DISCLOSURE

The disclosure relates to plant genetics. More particularly, the disclosure relates to methods for generating non-photosynthetic plant mutants and transforming mutant plant plastids by complementation of the non-photosynthetic defect or mutation.

### BACKGROUND

A plastid is a class of plant subcellular organelles that have evolved different functions depending on the tissue in which they arise. Chloroplasts are the best-known plastid type, and are specialized for chlorophyll production and active photosynthesis in green leaf cells. Other plastid types include root amyloplasts specialized for starch accumulation, chromoplasts in flower petals that are specialized for colored pigment development, and proplastids that are undeveloped precursors to other plastid types and reside in dark-grown embryogenic tissues often used in cell culture systems.

The plastid carries its own genome, which is a double-stranded circular DNA (dsDNA) of ˜155 kilobases encoding ˜110 genes. The plastid is a polyploid genetic system, and the number of plastids and plastid genomes differs depending on which cell type they reside in. For example, in leaf cells, chloroplasts and their DNA are abundant with up to ˜100 chloroplasts per cell and ˜100 dsDNAs per chloroplast, for a total of ˜10,000 DNAs per leaf cell.

Most of the genes encoded by the plastid genome are required for maintenance of the organelle itself (ribosomal RNAs, tRNAs, ribosomal proteins, etc). In addition, ˜35 plastid-encoded genes encode components of photosynthesis, with the large subunit gene (rbcL) of RUBISCO being the most well-studied. However, the vast majority of proteins that reside in the organelle are nuclear-encoded and imported into the organelle. Nuclear-encoded proteins include structural proteins, enzymes, and transcription/translation factors that help control expression of plastid-encoded genes.

The insertion of transgenes into the plastid genome was first achieved in tobacco in the 1990's. Success in plastid transformation of tobacco was facilitated by its routine tissue culture and transformation system that utilizes green leaf tissue with abundant chloroplasts as a recipient for transforming DNA. The favored selectable marker used in nearly all plastid transformation experiments is a bacterial-derived aadA gene that confers resistance to the antibiotics spectinomycin and streptomycin. Under antibiotic selection, sensitive green leaf cells will bleach and their growth will be inhibited (due to lack of plastid gene translation due to the antibiotic). Only transformed plastids that receive the selectable aadA marker gene will acquire resistance to the antibiotics and remain green and continue to grow normally. Plastid transformation has also been reported in the photosynthetic green algae, Chlamydomonas reinhardtii, which was facilitated by the availability of mutant strains that are non-photosynthetic due to deletion in one or more plastid-encoded genes required for photosynthesis. Deletion mutants were first created by treatment of cells with 5-fluoro-deoxy-uridine, a nucleotide base analog that causes disruption of plastid DNA replication and subsequent generation of deletion mutants.

### BRIEF SUMMARY OF THE DISCLOSURE

Therefore, a substantial need exists for a platform for precision engineering of agronomically beneficial traits into a plant chloroplast using plastid transformation via complementation of a non-photosynthetic defect or mutation in a nuclear gene. The objective of the present disclosure is to provide a method for transforming plant chloroplasts for use in complementation assays, as well as deletion mutants produced by such methods.

In one aspect, the disclosure provides a method of expressing an agronomically and/or non-agronomically beneficial trait in a plant plastid comprising: (a) utilizing a non-photosynthetic plant or plant part homozygous for the loss of function of a nuclear-encoded gene involved in photosynthesis in the chloroplast; (b) growing a callus of the non-photosynthetic plant in culture; (c) transforming mutant plant-line callus with a plastid transformation vector comprising a copy of the nuclear-encoded gene functional in plant plastids and an agronomically beneficial trait gene; and (d) selecting green, photosynthetic callus. In one embodiment, the plastid transformation vector comprises a chloroplast transformation vector. In another embodiment, the selection step is performed in light growth conditions. In another embodiment, the plant comprises a corn plant or a soy plant. In another embodiment, the recipient mutant plant line is a non-photosynthetic nuclear mutant plant line. In another embodiment, the mutant corn plant is a ppr10 mutant line, an atpc mutant or chli mutant line. In another embodiment, the corn ppr mutant line is ppr10-1 or ppr10-2. In another embodiment, the mutant soy plant is a chli mutant line or a psbP mutant line. In another embodiment, the mutant corn line is maintained in a hybrid genetic background. In another embodiment, the corn hybrid genetic background is A188×B73. In another embodiment, the disclosure provides a plant produced by a method as described herein. In another embodiment, the disclosure provides a plant part of a plant as described herein, selected from the group consisting of a seed, embryo, stem, callus, meristem, leaf, and root. In another embodiment, the disclosure provides a seed produced by a plant as described herein.

In another aspect, the disclosure provides a method of expressing an agronomically and/or non-agronomically beneficial trait in a plant comprising: (a) selecting a recipient mutant plant or callus homozygous for the loss of function of a nuclear-encoded gene involved in photosynthesis in the chloroplast; (b) growing a callus of the selected recipient mutant plant in culture; (c) transforming the mutant plant-line callus with a nuclear transformation vector comprising a wild-type copy of the nuclear-encoded gene and one or more agronomically and/or non-agronomically beneficial trait genes; (d) selecting green, photosynthetic callus in the light; and (e) regenerating a green plant and the agronomically and/or non-agronomically beneficial trait.

In another aspect, the disclosure provides a kit comprising: a single-use container comprising a callus or seed produced from a plant part as described herein. In one embodiment, the kit further comprises reagents for transformation, cell culture, or both.

In certain aspects, transplastomic plant cells comprising plastids containing one or more heterologous DNA insertion(s) into the genome of the plastids, wherein at least one of the heterologous DNA insertions encodes a protein having an enzymatic and/or biological activity of a nuclear gene which encodes a chloroplast-localized protein required for photosynthesis, and wherein the cell is photosynthetic. Also provided are whole plants, plant parts (e.g., seeds, leaves, tubers, roots, stems, or flowers), as well as callus and/or embryogenic tissue containing the aforementioned transplastomic plant cells, the use of the plants, plant cells, and tissues in agriculture, and methods of making the plant cells, plants, plant parts, and tissues.

Methods for transforming a plant plastid with a DNA molecule comprising: (a) introducing at least one DNA molecule comprising a first DNA sequence into a recipient non-photosynthetic plant cell comprising a loss-of-function mutation in each copy of a nuclear gene encoding a chloroplast-localized protein required for photosynthesis, wherein the DNA molecule encodes a protein having an enzymatic and/or biological activity of the chloroplast-localized protein required for photosynthesis; (b) exposing the transformed plant cell from step (a) to light sufficient to support greening of a photosynthetic plant cell; and, (c) selecting a green photosynthetic plant cell comprising a transformed plant plastid containing a plastid genome comprising the DNA molecule from the plant cells exposed to the light in step (b) are provided.

### BRIEF DESCRIPTION OF THE DRAWINGS

FIG. 1—Shows a construct containing a ScaI restriction enzyme site (psbC-trnG region), which serves as a site of transgene integration.

FIG. 2—Shows a construct containing DraI or NotI restriction enzyme sites (rbcL region), which serves as a site of transgene integration.

FIG. 3—Shows promoter/leader sequences and transcript stability sequences to drive expression of the modified PPR10 coding sequence as an example for expression in plastids and complementation of the nuclear-encoded non-photosynthetic phenotype, and GFP as a visual marker to help facilitate early identification of transformed lines. Similarly, the modified coding region of ATPC or CHLI can be used.

FIG. 4—Shows gene expression signals that are used to drive expression of the selectable marker gene and a GFP visual marker to help monitor plastid transformation at an early stage.

FIG. 5—Shows a flowchart demonstrating a protocol for antibiotic selection.

FIG. 6—Shows a flowchart demonstrating a protocol for restoration of photosynthesis.

FIG. 7—Shows a nuclear transgenic plant wherein green cells are selected by restoration of photosynthesis in a nuclear mutant plant.

FIG. 8A Developing green calli on MS3 medium with hygromycin (50 mg/L) under white light and FIG. 8B the same callus under fluorescent light showing green fluorescent from ZsGreen and chlorophyll autofluorescence (red) from complemented tissues.

### BRIEF DESCRIPTION OF SEQUENCES

SEQ ID NO:1—Mature PPR10 amino acid sequence lacking a chloroplast transit peptide (CTP) with methionine start codon and alanine added).

SEQ ID NO:2—Mature PPR10 nucleotide sequence (lacking CTP, with ATG methionine start codon added and GCG Alanine codon added).

SEQ ID NO:3—Maize genome homology region 1 (psbC-trnG).

SEQ ID NO:4—Maize rbcL homology region.

SEQ ID NO:5—Maize plastid ClpP promoter and ClpP leader (PclpP-LclpP).

SEQ ID NO:6—Maize plastid ClpP promoter-ClpP leader-Phage T7 gene 10 ribosome binding site.

SEQ ID NO:7—Maize Prrn promoter G10L+10 amino acids of GFP.

SEQ ID NO:8—Maize Prrn 16s rDNA promoter-maize ClpP promoter-ZmClpP leader.

SEQ ID NO:9—Maize petD gene terminator ZmTpetD.

SEQ ID NO:10—Tobacco rps16 gene terminator Trps16.

SEQ ID NO:11—Tobacco psba gene terminator TpsbA (short).

SEQ ID NO:12—E. coli terminator from rrnB gene (E coli TrmB).

SEQ ID NO:13—Sequence of aadA.

SEQ ID NO:14—Sequence of nptII.

SEQ ID NO:15—Sequence of aphA-6.

SEQ ID NO:16—Sequence of PPR10F forward primer for amplification of PPR10 gene.

SEQ ID NO:17—Sequence of PPR10R reverse primer for amplification of PPR10 gene.

SEQ ID NO:18—Sequence of mu1 primer for amplification of PPR10 gene that is disrupted by a Mu transposon.

SEQ ID NO:19—Sequence of ATPC plastid gene amino acid sequence (lacking CTP, with ATG methionine start and alanine codons added).

SEQ ID NO:20—Sequence of ATPC plastid gene nucleotide sequence (lacking CTP, with ATG methionine start and GCT alanine codons added).

SEQ ID NO:21—Sequence of CHLI plastid amino acid sequence (lacking CTP, with ATG methionine start and alanine codons added).

SEQ ID NO:22—Sequence of CHLI plastid gene nucleotide sequence (lacking CTP, with ATG methionine start and GCT alanine codons added).

SEQ ID NO:23—Sequence of ATPCF forward primer.

SEQ ID NO:24—Sequence of ATPCR reverse primer.

SEQ ID NO:25—Sequence of CHLIF forward primer.

SEQ ID NO:26—Sequence of CHLIR reverse primer.

SEQ ID NO:27—Sequence of Maize PPR10 amino acid.

SEQ ID NO:28—Maize PPR10 cDNA sequence.

SEQ ID NO:29—Zea mays cultivar B73 chromosome 7, B73 RefGen_v4, whole genome shotgun sequence.

SEQ ID NO:30—ATP synthase subunit gamma, chloroplastic precursor

SEQ ID NO:31—Maize ATPC cDNA sequence.

SEQ ID NO:32 Maize CHLI.

SEQ ID NO:33—Maize CHLI amino acid sequence.

SEQ ID NO:34—Maize CHLI cDNA sequence.

SEQ ID NO:35—Soybean psbP cDNA sequence.

SEQ ID NO:36—Soybean CHLI1a genomic sequence.

SEQ ID NO:37—Glycine max Mg-protoporphyrin IX chelatase subunit ChlI (CHLI), mRNA.

SEQ ID NO:38—Magnesium-chelatase subunit ChlI, chloroplastic [Glycine max].

SEQ ID NO:39—CHLI1b; GenBank: MG696843.1>MG696843.

SEQ ID NO:40—Glycine max cultivar Williams 82 chloroplast magnesium chelatase I subunit (ChlI1b) amino acid sequence.

### DETAILED DESCRIPTION

Embodiments of the present disclosure provide methods of expressing an agronomically and/or non-agronomically beneficial trait in a plant plastid comprising: (a) utilizing a non-photosynthetic plant or plant part homozygous for the loss of function of a nuclear-encoded gene involved in photosynthesis in the chloroplast; (b) growing a callus of the non-photosynthetic plant in culture; (c) transforming mutant plant-line callus with a plastid transformation vector comprising a copy of the nuclear-encoded gene functional in plant plastids and an agronomically and/or non-agronomically beneficial trait gene; and (d) selecting green, photosynthetic callus. The non-photosynthetic plant is then grown as callus tissue and used as a recipient plant for complementation studies in which a gene conferring an agronomically and/or non-agronomically beneficial trait is introduced into the non-photosynthetic recipient plant plastids along with a functional copy of the mutated photosynthetic gene. A plant that is able to perform photosynthesis following complementation studies indicates that the plant plastid received the functional copy of the photosynthetic gene and by extension, the gene conferring the agronomically and/or non-agronomically beneficial trait. Other embodiments provide for mutations in a nuclear-encoded gene whose products are targeted to the chloroplast and required for photosynthesis. Other embodiments of the disclosure provide for plants produced by these methods, as well as plant parts. Elements, plants, reagents, or components used in the methods as described herein may also be provided in the form of a kit for introducing an agronomically and/or non-agronomically beneficial trait to a plant as described herein.

Large-scale forward genetic screens for mutants in photosynthesis have been performed in maize and other plant species, using pigment mutations, including albino, pale green, yellow, etc., as the observable phenotype (see, for example, Belcher et al., 2015). The Photosynthetic Mutant Library (PML) is a recent collection of 2000 independently arising mutants that were selected from Mu-transposon-activated maize lines based on seedling chlorophyll deficiency. The majority of mutants are seedling lethal and die at around 3 weeks post-germination. The mutations are all nuclear-encoded and recessive, and thus mutant lines can be maintained as heterozygotes. Those skilled in the art will recognize that numerous nuclear-encoded genes have control over plastid-encoded photosynthetic gene function. Mutation in any of these nuclear genes may result in non-photosynthetic plant lines that are very pale green or albino in tissue culture but can survive on media that does not require photosynthesis for growth. Likewise, because of the conservation of chloroplast gene functions, similar mutants may exist in other monocot or dicot species.

PML mutants may also arise from mutation in a large variety of nuclear-encoded proteins that are imported into chloroplasts. These include proteins involved in import of nuclear-encoded proteins into the organelle, structural components of photosynthesis (i.e., RUBISCO, PSI, PSII, ATP Synthase, etc.) and transcription or translation factors required for expression of plastid-encoded genes.

Analysis of some members of the PML group of mutants resulted in identification of the Pentricopeptide Repeat (PPR) class of proteins that act in various aspects of RNA processing and translation of plastid- and mitochondrial-encoded genes. PPR proteins are a large family of proteins with more than 400 members in higher plant species. PPR proteins are characterized by 2 to around 30 tandem repeats of a helical amino acid repeat motif that binds to single-stranded RNA in a sequence-specific fashion. Computational methods have inferred a code for nucleotide recognition involving 2 amino acids in each PPR repeat. The “PPR code” (Barkan et al., 2012) is a direct readout of the RNA binding site for the PPR protein. It is therefore possible to identify the candidate plastid-encoded genes that are putatively regulated by each PPR protein simply by knowing the amino acid sequence of the PPR protein and the sequence of the plastid genome.

PPR proteins can be divided into 2 classes, denoted P class and PLS class. P class members carry a helical tandem repeat of 35 amino acids and are implicated in RNA stabilization, processing, splicing, and translation. PLS class members participate in RNA editing, which is common in chloroplasts and mitochondria.

In another example, ATP4 is a nuclear-encoded PPR protein that has been described in maize (Zoschke et al., 2012), which binds to the 5′-untranslated region (UTR) of the atpB/E operon mRNA and is required for translation of the atpB gene. Although ATP4 may enhance expression of other plastid genes, mutation of this gene causes a non-photosynthetic phenotype due mainly to lack of atpB mRNA translation. In this case, the defect in photosynthesis may be corrected by either of two approaches: (1) expression of the mature ATP4 protein from within the plastid, or (2) replacement of the resident plastid atpB 5′-UTR with a new sequence that eliminates the need for binding of ATP4 for translation. In the latter case, for example, the atpB 5′UTR may be replaced with a synthetic ribosome binding site region, such as the bacteriophage gene 10 leader (G10L), which has previously been used to allow constitutive translation of multiple plastid transgenes. Plastid transformation would utilize an engineered atpB/E operon sequence carrying G10L instead of the resident sequence, and additional plastid transgenes would be integrated alongside the atpB operon. In both approaches, selection of transformed tissue would by identifying green photosynthetic tissue in light-grown callus on appropriate medium.

PPR10 protein was first identified in maize, and is a P class PPR protein with 19 PPR motifs. PPR10 is nuclear-encoded and targeted to plastids by a N-terminal chloroplast transit peptide (CTP) that is cleaved off upon entry into the organelle. PPR10 protein binds to 2 similar sequences in the maize chloroplast genome, near the 5′ end of the atpH coding region and in the 3′ end of psaJ plastid gene mRNA (Pfalz et al., 2009). In the case of atpH, PPR10 binding stimulates translation of the mRNA while for psaJ, PPR10 binding stabilizes the mRNA. Mutants in PPR10 are non-photosynthetic and homozygous seedlings will survive for only about two weeks in soil. Heterozygous plants are therefore used to maintain the mutant.

Homozygous PPR10 lines can be maintained in tissue culture as callus. If maintained on a medium that requires photosynthesis, then callus grown in the light is expected to be non-green or very pale-green. Plastid transformation can be attempted by expression in plastids of the mature PPR10 protein, without its N-terminal chloroplast transit peptide. Plastid expression can be achieved by a variety of typical plastid gene expression signals, and integration of the PPR10 gene into the plastid genome can be achieved using a variety of standard transformation vectors that target the transgene to an intergenic region or other non-coding region of the maize plastid genome. Most importantly, selection for photosynthetic ability would be attempted, in the light on medium where greening requires active photosynthesis. Plastid transformed callus would be expected to turn green while non-transformed callus would remain colorless or very pale-green. Greening callus can be sub-cultured to amplify tissue to homoplasmy or plants can be regenerated using standard methods.

In some embodiments, the present disclosure provides a method of expressing an agronomically and/or non-agronomically beneficial trait in a plant comprising: (a) selecting a recipient mutant plant or callus homozygous for the loss of function of a nuclear-encoded gene involved in photosynthesis in the chloroplast; (b) growing a callus of the selected recipient mutant plant in culture; (c) transforming the mutant plant-line callus with a nuclear transformation vector comprising a wild-type copy of the nuclear-encoded gene and one or more agronomically and/or non-agronomically beneficial trait genes; (d) selecting green, photosynthetic callus in the light; and (e) regenerating a green plant and the agronomically and/or non-agronomically beneficial trait. In some embodiments, a selectable marker useful for testing nuclear transformation may use one of the plant's natural nuclear genes. Such a selectable marker may eliminate the use of selectable markers utilizing herbicides or antibiotics, which have typically been used for nuclear transformants, and which may be undesirable in the final product. A method as described herein would thus simply restore the normal nuclear gene, eliminating any extra genetic material or “extra genes.”

### CHLI (Magnesium Chelatase Subunit I)

Mutations in genes involved in chlorophyll biosynthesis were also identified in the maize PML mutant collection. For example, mutation in any subunit (CHLI, CHLD or CHLH) of the multi-subunit chlorophyll biosynthesis enzyme, magnesium chelatase, may result in a non-photosynthetic phenotype. A mutant maize line was obtained that carried a non-photosynthetic mutation in subunit I of this enzyme (Magnesium chelatase subunit I). Homozygous mutants are albino or very pale yellow because chlorophyll is not produced in these lines. Heterozygous plants are used to maintain seeds and homozygous seedlings are identified by their albino phenotype. These seedlings will die in soil after a couple of weeks, but embryogenic cultures derived from immature embryos of homozygous seeds can be cultured in vitro to form callus. Callus is non-green but viable on medium containing sugars.

Mutations in magnesium chelatase that result in the non-photosynthetic phenotype exist in other monocots and dicots. In other monocots, for example, these include the rice chlorina-1, ygl98, and ygl3 mutants in magnesium chelatase subunit D and the rice chlorina-9 mutation in magnesium chelatase subunit I, and barley Xantha and chlorina mutants in the CHLI subunit (reviewed in Sandhu et. al.; 2018). In dicots, similar mutants exist in cotton (Zhu et al; 2017) and cucumber (Gao et. al, 2016).

### Soybean

Nuclear-encoded non-photosynthetic mutants can also be obtained in soybean and several mutants have been identified by their yellow or chlorophyll-deficient foliage. In spite of their widespread occurrence, only a few of the nuclear genes responsible for these mutants have been identified at the molecular level (reviewed in Sandhu et al; 2018). For example, the yl1 and the CD-5 mutant affects the paralogous soybean Chli1a and Chli1b subunit genes of soybean magnesium chelatase, respectively. The well characterized MinnGold mutant is an example of a different mutant allele in the Chli1a gene (Campbell et al., 2015). Photosynthesis is affected directly in the T378H line that carries a mutation in the psbP gene, resulting in >90% loss of chlorophyll from this line (Sandhu et al., 2016).

Thus, in some embodiments, the disclosure provides methods of expressing an agronomically and/or non-agronomically beneficial trait in a plant plastid comprising: utilizing a non-photosynthetic plant homozygous for the loss of function of a nuclear-encoded gene; growing a callus of the non-photosynthetic plant in culture; transforming mutant plant-line callus with a plastid transformation vector comprising a wild-type copy of the nuclear-encoded gene functional in plant plastids and an agronomically and/or non-agronomically beneficial trait gene; and selecting green, photosynthetic callus. A number of genes required for photosynthesis are produced by the chloroplast genome. However, there are a number of proteins and/or gene products that are required for photosynthesis that are encoded by the nuclear genome of a plant cell. Nuclear-encoded genes required for photosynthesis are targeted to the chloroplast with the use of a CTP, as described herein. CTPs are present on a translated protein, which enables their entry into the plant chloroplast. The CTP is then removed from the protein by the chloroplast processing enzyme. CTPs may be naturally occurring in a gene to be transported to the chloroplast, or they may be artificially engineered and linked to a nucleic acid encoding a particular protein to target the protein to the chloroplast.

Nuclear genome encoded genes required for photosynthesis that can be targeted for disruption and/or complementation by the methods provided herein include, but are not limited to, the maize genes set forth below in Table 1, as well as functional equivalents thereof (e.g., genes encoding proteins have an enzymatic and/or biochemical activity of the gene set forth in Table 1) found in monocot (e.g., corn, wheat, rice, or sorghum) or dicot crop plants (e.g., soybean, cotton, tomato, or potato).

TABLE 1
Nuclear-encoded genes conferring a non-photosynthetic phenotype when mutated.

| Gene | Maize locus ID | Gene function |
|---|---|---|
| crp1 | GRMZM2G083950 | PPR protein; Activates petA and psaC translation; |
| | | stabilizes processed petB, petD mRNAs; binds |
| | | petA, psaC, petD 5′UTRs |
| ppr10/luteus15 | GRMZM2G177169 | PPR protein; activates atpH translation, stabilizes |
| | | atpH and psaJ mRNAs, binds atpI-atpH and psaJ- |
| | | rpl33 intergenic regions |
| atp4 | GRMZM2G128665 | PPR-SMR protein; activates atpB translation, |
| | | binds atpB 5′UTR |
| tha8 | AC217965.2_FG012 | PPR protein; required for splicing ycf3-int2 and |
| | | trnA introns and associates with those introns in |
| | | vivo |
| Zm-otp51 | GRMZM2G325019 | PPR-LAGLIDADG protein; required for splicing |
| | | cp ycf3-int2 |
| crs1 | GRMZM2G078412 | cp RNA splicing 1; founding member of CRM |
| | | domain family. Binds atpF intron, promotes atpF |
| | | splicing |
| crs2 | GRMZM2G132021 | cp RNA splicing 2; required for splicing of |
| | | chloroplast subgroup IIB introns. peptidyl tRNA |
| | | hydrolase homolog. |
| rnc1 | GRMZM2G035820 | RNAse III domain protein required for the |
| | | splicing of many cp introns. Interacts with WTF1. |
| wtf1 | GRMZM2G403797 | cp RNA splicing factor. Founding member of |
| | | PORR domain family. Interacts with RNC1. |
| Zm-mterf4 | GRMZM2G029933 | cp mTERF domain protein. Binds in vivo to |
| | | multiple group II introns and promotes their |
| | | splicing. |
| crp4 | GRMZM2G377761 | cp polynucleotide phosphorylase, cpRNA |
| | | processing and decay |
| Zm-ptac12 | GRMZM5G897926 | Maize ortholog of PTAC12; PEP associated |
| | | protein required for PEP-mediated transcription |
| Zm-ptac2 | GRMZM2G122116 | Maize ortholog of PTAC2; PEP associated protein |
| | | required for PEP-mediated transcription |
| Zm-murE | GRMZM2G009070 | Maize ortholog of MurE; PEP associated protein |
| | | required for PEP-mediated transcription |
| Zm-prin2 | GRMZM2G119906 | Maize ortholog of PRIN2; PEP associated protein |
| | | required for PEP-mediated transcription |
| Zm-hcf107 | GRMZM2G121960 | HAT domain protein required to stabilize psbH 5′ |
| | | end and promote psbH translation. Binds psbH |
| | | 5′UTR |
| Zm-why1 | GRMZM2G155662 | single-stranded nucleic acid binding protein. |
| | | Multifunctional: promotes atpF splicing, 23S |
| | | rRNA maturation, stabilizes cp genome. |
| rlsb1 | GRMZM2G087628 | cp RNA binding protein |
| hcf106 | GRMZM5G898735 | thylakoid TAT transport |
| csr1 | NP_001105732 | CpFtsY. cp SRP receptor involved in thylakoid |
| | (missing from B73 v3 | protein targeting |
| | genome) | |
| csy1 | GRMZM5G809546 | CpSecY. thylakoid protein translocase |
| pet2 | GRMZM2G087063 | Ortholog of HCF208/CCB2; cyt b6 heme |
| | | attachment factor |
| psa2 | GRMZM2G021687 | DnaJ-type zinc finger domain protein required for |
| | | PSI assembly |
| raf1 | GRMZM2G457621 | Rubisco Accumulation Factor1; promotes Rubisco |
| | | assembly |
| raf2 | GRMZM2G139123 | Rubisco Accumulation Factor2; promotes Rubisco |
| | | assembly |
| bsd2 | GRMZM2G062788 | bundle sheath defective 2. Rubisco assembly |
| | | factor |
| g2/bsd1 | GRMZM2G087804 | golden2. Nuclear transcription factor for |
| | | chloroplast biogenesis genes |
| Zm-hcf136/ | GRMZM2G102838 | PSII assembly factor |
| psb1* | | |
| elm2 | GRMZM2G101004 | elongated mesocotyl2. heme oxygenase4 |
| cpx1/nec4* | GRMZM5G870342 | coproporphyrinogen III oxidase |
| ppr8522/emb2 | GRMZM5G884466 | cp PPR protein, plastid gene expression |
| oy1/chli | GRMZM2G419806 | Chlorophyll synthesis, Magnesium chelatase I |
| Zm-prda1 | GRMZM2G079452 | Ortholog of Arabidopsis PRDA1. cp nucleoid |
| | | protein involved in PEP-mediated transcription; |
| | | cpRNA defects in maize similar to other PEP- |
| | | deficient mutants |
| Zm-ptac14 | GRMZM5G807767 | Ortholog of Arabidopsis PTAC14. PEP-associated |
| | | protein, required for PEP-mediated transcription; |
| | | cpRNA defects in maize similar to other PEP- |
| | | deficient mutants |
| Zm-pgr3 | GRMZM2G372632 | Ortholog of Arabidopsis PGR3. PPR protein that |
| | | stabilizes 5′-end of cp petL RNA and is required |
| | | for NDH accumulation. Additional functions in |
| | | maize. |
| Zm-ccs1 | GRMZM2G038301 | Ortholog of CCS1. Required for cytb₆f of assembly |
| pet3 | GRMZM2G177145 | Ortholog of CCB4. Required for cyt b₆ assembly |
| psa1 | GRMZM2G100976 | Required for PSI accumulation. Harbors NifU |
| | | domain implicated in Fe-S cluster assembly. |
| | | Ortholog of Arabidopsis Nfu-IVa. |
| cps2 | AC215201.3_FGP005 | cpn60 α1. Molecular chaperone involved in |
| | | Rubisco assembly. Also affects distribution of |
| | | ribosomes on chloroplast mRNAs. |
| Zm-y3IP1 | GRMZM2G002165 | Ortholog of Arabidopsis Y3IP1. PSI assembly |
| | | factor, cooperates with Ycf3 |
| Zm-gun4 | GRMZM2G464328 | Ortholog of Arabidopsis GUN4; Mg-chelatase |
| | | cofactor and porphyrin binding protein |
| prpo1/Zm-ppox | GRMZM2G039396 | Ortholog of Arabidopsis PPOX/PPO1. |
| | | protophorphyrinogen oxidase |
| Zm-atpc | GRMZM2G048907 | gamma subunit of chloroplast ATP synthase |
| | | (ATPC1) |

Sequences associated with the Maize Locus ID can be accessed on the world wide web internet site “maizegdb.org/gene_center/gene.”

In certain embodiments, germplasm comprising the aforementioned mutants can be accessed from repositories and/or academic laboratories. In some embodiments, any method of inactivation, disruption, or mutation of a gene, in particular a nuclear-encoded gene, may be employed in accordance with the disclosure. For example, non-limiting examples of methods for such gene inactivation may include transposon mutagenesis, such as was used for the PML mutant library described herein. Alternatively, transposon mutagenesis, chemical mutagenesis, radiation mutagenesis, homologous recombination, or any other appropriate means for disruption of a gene or its function may be used in accordance with the disclosure.

Transposon mutagenesis is well-known in the art and widely used to induce mutation in genes encoding a variety of functions. For example, one non-limiting example of a transposon useful in accordance with the disclosure is the result of the activity of the mu bacteriophage, or “mu” as described in detail herein. However, one of skill in the art will recognize that any transposon capable of causing a mutation or disruption in a gene of interest may be used and is therefore encompassed within the scope of the present disclosure.

In some embodiments, a recipient plant as described herein may have a mutation in a PPR gene. There are a large number of PPR genes in plants, any of which may be mutated within the scope of the present disclosure. A plant having a mutation in a PPR gene may be referred to herein as a “PPR mutant plant” or a “PPR mutant line.” For example, a PPR mutant line in accordance with the disclosure may include, but is not limited to, a ppr10 mutant line, such as ppr10-1 or ppr10-2. Mutant lines as described herein, such as ppr mutant lines, may be produced using the methods of the disclosure, and may be used as recipient plant lines for complementation studies as described herein.

In some embodiments, cells from the non-photosynthetic recipient plant may be cultured under sterile tissue culture conditions to produce callus tissue. The non-photosynthetic callus tissue may then be provided with one or more nucleic acid molecules such that the non-photosynthetic trait is complemented with the introduced nucleic acid. In some embodiments, providing the cell with a functional copy of the nuclear-encoded gene enables genetic complementation in the chloroplast rather than the nucleus, as the functional protein is imported into the chloroplast. For example, providing the recipient plant with a functional copy of the mutated nuclear-encoded gene (i.e., PPR, ATPC or CHLI gene) in the chloroplast genome may be desired in some embodiments, and may genetically complement the non-photosynthesis trait in the chloroplast. In other embodiments, a functional copy of the mutated nuclear-encoded gene may be provided to the recipient chloroplast genome along with an agronomically and/or non-agronomically beneficial trait gene as desired.

In some embodiments, a nucleic acid encoding a functional copy of a mutated nuclear-encoded gene involved in photosynthesis may be provided to the non-photosynthetic mutant plant callus in order to complement the non-photosynthetic trait and result in a plant callus that is able to perform photosynthesis and is therefore green in color. A gene conferring an agronomically and/or non-agronomically beneficial trait may be provided to the non-photosynthetic mutant plant callus along with the functional copy of the nuclear-encoded photosynthesis gene. As described above, nucleic acids may be provided to a plant or plant tissue in a vector or other vehicle or delivery system. For example, in some embodiments, a transformation vector such as a plastid transformation vector may be used as appropriate. In some embodiments, a plastid transformation vector may comprise a chloroplast transformation vector. One of skill in the art will understand and be able to select a beneficial delivery system for use with the present disclosure.

To ensure that the functional copy of the nuclear-encoded photosynthesis gene, and/or gene conferring an agronomically and/or non-agronomically beneficial trait is expressed in the plant as desired, these elements may be operably linked to a promoter functional in plants. For example, a promoter useful with the exogenous nucleic acid may be a constitutive promoter, a tissue-specific promoter, a developmentally regulated promoter, or a cell cycle regulated promoter. Certain contemplated promoters may include ones that only express in the germline or reproductive cells, among others. In accordance with the disclosure, a promoter as described herein may be a seed-specific promoter or an embryo-specific promoter. Any appropriate promoter may be used as long as the promoter is functional in plants.

Expression of a nuclear-encoded photosynthesis gene in the chloroplast, and an agronomically and/or non-agronomically important gene in the chloroplast, would be accomplished using a promoter functional in plant plastids. Promoters functional in plant plastids include the promoter of a native plastid gene. In some cases, a bacterial-derived promoter may be used, such as a promoter from E. coli or a bacteriophage that is capable of replication in E. coli.

As described herein, a plant useful for any of the methods of the disclosure may comprise any type of plant appropriate for the particular application, or to which a desirable or agronomically and/or non-agronomically beneficial trait is to be added. Any crop or ornamental plant may be used, such as including, but not limited to, soybean, corn, potato, wheat, or the like. In some specific embodiments, the plant may be, for example, a corn plant or a soy plant. One of skill in the art will understand how to modify a method of the disclosure to accommodate a particular plant as needed.

In some embodiments, a plant in which a gene has been interrupted may be referred to herein as a “recipient non-photosynthetic plant cell,” “recipient mutant plant cell or plant line,” or a “recipient plant cell or plant line” or a “recipient.” Such a plant or plant cell may, in accordance with certain embodiments, have a mutation in a nuclear-encoded photosynthetic gene such that the recipient plant or plant cell cannot perform photosynthesis under light conditions at wild-type levels and will have a non-green phenotype. By “non-green” is meant a plant that is deficient, completely or in part, in photosynthesis, and therefore cannot produce chlorophyll, leading to an absence or reduction of green pigmentation in the plant. Such plants may also be referred to herein as “non-photosynthetic.” Non-photosynthetic “loss-of-function” mutations thus include mutations which confer a complete or partial loss-of-function of the gene (e.g., 0%, 5%, 10%, 20%, 30%, or 40% or less of wild-type activity; or alternatively, 0%, 5%, or 10% to 15%, 20%, or 40% of wild-type activity). A non-photosynthetic plant as described herein may be any level of non-green, i.e., white, pale green, yellow-green, or the like. “Non-green” refers to a phenotypic color of the plant tissue that has less green pigment (i.e., is less green) that a wild-type plant that is able to perform photosynthesis.

In some embodiments, a non-photosynthetic plant as described herein may be a homozygous non-photosynthetic mutant, having both non-functional copies of the nuclear-encoded photosynthesis gene. In other embodiments, it may be beneficial to perform certain steps of the methods described herein in a heterozygous plant. In some embodiments, a non-photosynthetic mutant plant may also be a homoplasmic chloroplast mutant plant line. As used herein, “homoplasmic” refers to a plant in which all copies of the chloroplast or plastid genome are identical. Homoplasmy may occur naturally in a plant, or it may be artificially induced using methods known in the art. One or more mutations that result in a non-photosynthetic plant may be introduced by any methods known in the art.

As described herein, a gene present in the nuclear genome but required for photosynthesis may be modified, interrupted, mutated, altered, eliminated, etc., using genetic engineering methodology, which is well-known in the art. Such a plant may then be used as a recipient for complementation studies wherein a functional copy of a mutated nuclear gene is introduced into the plant plastid along with a gene conferring a trait of interest, such as an agronomically and/or non-agronomically beneficial trait. In this way, introduction of the functional nuclear-encoded gene and the gene of interest into the recipient plant chloroplast may restore photosynthesis in the recipient plant, as well as provide the desired agronomically and/or non-agronomically beneficial trait to the plant. Thus, in some embodiments, complementation as described herein may be used as a marker of integration of a trait of interest into a recipient plant. Such a marker may eliminate the need for a further selection step, such as antibiotic selection to identify effectively transformed plants, referred to herein as “transformants.”

In some embodiments, a mutant line as described herein may be maintained in a particular genetic background, in order to maintain plants with the desired genetic mutation. For example, as described herein, a mutant line may be maintained in a hybrid genetic background. Plants heterozygous for a genetic mutation as described herein may carry the non-photosynthetic mutation while exhibiting a photosynthetic phenotype. Plant breeding programs may be used to maintain a desired genetic mutation in a particular plant line or genetic background. Plant breeding programs are well-known in the art and can be modified as necessary depending on the growth requirements of the particular plant species used. For example, in some embodiments and as appropriate for the particular mutation, whether chloroplast-encoded or nuclear encoded, a mutation producing a non-photosynthetic mutant plant in accordance with the disclosure may be maintained in a hybrid genetic background such as including, but not limited to, an A188×B73 hybrid genetic background. In other embodiments, the a non-photosynthetic mutant plant in accordance with the disclosure may be maintained in inbred genetic backgrounds including elite inbred genetic backgrounds Any genetic background may be used in accordance with the disclosure, as long as the desired mutation is able to be maintained for the desired use.

In some embodiments, a nucleic acid encoding a functional copy of a mutated photosynthesis gene may be provided to the non-photosynthetic mutant plant callus in order to complement the non-photosynthetic trait and result in a plant callus that is able to perform photosynthesis and is therefore green in color. A gene conferring an agronomically and/or non-agronomically beneficial trait may be provided to the non-photosynthetic mutant plant callus along with the functional copy of the photosynthesis gene. As described above, nucleic acids may be provided to a plant or plant tissue in a vector or other vehicle or delivery system. For example, in some embodiments, a transformation vector such as a plastid transformation vector may be used as appropriate. In some embodiments, a plastid transformation vector may comprise a chloroplast transformation vector. One of skill in the art will understand and be able to select a beneficial delivery system for use with the present disclosure.

As used herein, a “plastid transformation vector” or “chloroplast transformation vector” refers to a vector for transformation of a plastid as described herein. For example, a plastid transformation vector may be used as described herein for transformation of one or more chloroplasts of a plant having a non-photosynthetic phenotype. In such cases, the plastid transformation vector may be referred to as a chloroplast transformation vector. In accordance with the disclosure, a plastid transformation vector and a chloroplast transformation vector may be used interchangeably. As described herein, a chloroplast transformation vector may have a functional copy of a gene that was inactivated, mutated, or disrupted for expression in a non-photosynthetic mutant plant as described herein. A chloroplast transformation vector useful in accordance with the disclosure may also have at least a second gene for expression in a non-photosynthetic mutant plant as described herein, which may confer an agronomically and/or non-agronomically beneficial trait to the plant. In such a way, transformation of a non-photosynthetic plant may result in or produce a photosynthetic plant having an added agronomically and/or non-agronomically beneficial trait. Such traits are described herein elsewhere.

To ensure that a nucleic acid, or a functional copy of a photosynthesis gene, and/or a gene conferring an agronomically and/or non-agronomically beneficial trait is expressed in the plant as desired, the introduced elements may be operably linked to a promoter functional in plants. Any appropriate promoter may be used as long as the promoter is functional in plants or plant plastids.

In some embodiments, a plant useful for the disclosure may comprise any type of plant appropriate for the particular application, or to which a desirable or agronomically and/or non-agronomically beneficial trait is to be added. For example, a plant may be any crop or ornamental plant, such as including, but not limited to, soybean, corn, potato, wheat, or the like. In some embodiments, the plant may be a monocot or a dicot species. Some embodiments provide particular benefit to transformation of monocot species, which have thus far lacked effective methods for plastid transformation. Monocot species that may be particularly useful may include corn, wheat, rice, sorghum, Asparagus, sugarcane, onion, garlic, or the like. In some embodiments, the disclosure may be useful for transformation of plastids, e.g., chloroplasts, in a monocot plant. In some specific embodiments, the plant may be, for example, a corn plant or a soy plant. One of skill in the art will understand how to modify a method of the disclosure to accommodate a particular plant as needed.

In some embodiments, a plant useful in accordance with the present disclosure may have a gene that has been interrupted or mutated such that the plant may be used to determine complementation of the gene. Such a plant may be referred to herein as a “recipient mutant plant line” or a “recipient plant line” or a “recipient.” Such a plant may, in accordance with the disclosure, have a mutation in a photosynthetic gene such that the recipient plant cannot perform photosynthesis under light conditions and will have a non-green phenotype. By “non-green” is meant a plant that is deficient, completely or in part, in photosynthesis, and therefore cannot produce chlorophyll, leading to an absence of green pigmentation in the plant. Such plants may also be referred to herein as “non-photosynthetic.” Non-photosynthetic “loss-of-function” mutations in nuclear-encoded photosynthetic genes thus include mutations which confer a complete or partial loss-of-function of the gene (e.g., 0%, 5%, 10%, 20%, 30%, or 40% or less of wild-type activity; or alternatively, 0% or 5%, or 10%, 15%, 20%, or 40% of wild-type activity). For example, as described herein, a mutation in a chloroplast gene confers a non-green (i.e., non-photosynthetic) phenotype when grown under light conditions. A non-photosynthetic plant as described herein may be any level of non-green, i.e., white, pale green, yellow-green, or the like. “Non-green” refers to a phenotypic color of the plant tissue that has less green pigment (i.e., is less green) that a wild-type plant that is able to perform photosynthesis.

In some embodiments, non-photosynthetic mutants as described herein may be maintained on a rich media source. Any rich media source appropriate for growth of plant tissue or cells may be used. In some embodiments, non-photosynthetic mutants as described herein can be maintained on a media which comprises a sugar, an organic acid, or a combination thereof that supports growth of the whole plant, whole plant seedling, or whole plant part. In some embodiments, a non-photosynthetic mutant may not grow on media that requires active photosynthesis for survival. In other embodiments, a non-photosynthetic mutant may grow poorly on media that requires active photosynthesis for survival.

In some embodiments, a plant as described herein may be grown under dark conditions, referring to conditions under which photosynthesis will not normally occur. In other embodiments, a plant as described herein may be grown under light conditions. Light conditions as used herein is in reference to conditions under which photosynthesis would normally occur. As would be understood by one of skill in the art, growth conditions such as lighting may be altered to suit a particular plant species or to take advantage of the needs of a particular species.

For example, as described herein, some plants having a mutation in a gene required for photosynthesis, either plastid-encoded or nuclear-encoded, will have altered function as compared to a wild type plant. In a particular, non-limiting example, a plant having a mutation in a photosynthesis gene, referred to herein as a non-photosynthetic mutant, may be unable to perform photosynthesis under typical high-light conditions usually used for growth of wild-type plants. In accordance with the disclosure, growth conditions may be altered as necessary for the particular non-photosynthetic mutant. For example, a particular non-photosynthetic mutant may still be able to accumulate chlorophyll and thus have a reduced level of green color under dim light conditions. Such mutant plant lines may grow slowly under dim light, but would grow more poorly and begin to bleach and lose green color if transferred from dim light to a bright light on media requiring photosynthesis. Such a method may allow non-photosynthetic mutant plant lines to grow and amplify enough to perform plastid transformation experiments. In contrast, wild-type cells would be expected to grow fast and turn green under both high-light and dim-light conditions. Alternatively, non-photosynthetic mutant lines may be grown in the dark as typical callus to amplify material for plastid transformation experiments. Such mutant plant lines may then be shifted into the light to select for plastid transformed plants where photosynthesis is required for growth. Non-photosynthetic mutant callus would remain non-green in high-light or only slowly turn green under dim light, whereas plastid transformed cells would turn fully green under these same conditions. In contrast, wild-type callus would turn green when shifted from the dark to any light condition. In some embodiments, a non-photosynthetic plant as described herein may be a mutant plant line with a mutation in a gene involved in photosynthesis. Such a plant may, as a result of the lack of photosynthesis, be non-green.

As described herein, a nuclear gene involved in photosynthesis may be mutated, deleted, modified, or otherwise altered such that the gene is non-functional, resulting in a non-photosynthetic plant. Such a plant or plant part may then be used as a recipient for complementation studies wherein a functional copy of a mutated nuclear gene is introduced into the plant plastid along with a gene conferring a trait of interest, such as an agronomically and/or non-agronomically beneficial trait. In this way, introduction of the functional nuclear gene and the gene of interest into the recipient plant plastid may restore photosynthesis in the recipient plant, as well as provide the agronomically and/or non-agronomically beneficial trait to the plant. Thus, in some embodiments, complementation as described herein may be used as a marker of integration of a trait of interest into a recipient plant. Such a marker may eliminate the need for a further selection step, such as antibiotic selection to identify effectively transformed plants, referred to herein as “transformants.”

In another example, when mutant non-photosynthetic cultures are used, the cultures are already non-green or pale-green on media that requires photosynthesis for greening. In this case, the mutant may be complemented using expression of the nuclear gene in chloroplasts or the restoring chloroplast gene in chloroplasts and therefore the selection step is for photosynthesis, growth, and green on media that requires photosynthesis. The “selectable marker,” then, is the nuclear gene or the restoring chloroplast gene.

In some embodiments, a recipient plant as described herein may have a mutation in any nuclear-encoded photosynthesis gene whose protein is subsequently imported into the chloroplast genome. There are a large number of nuclear-encoded chloroplast genes in plants, any of which may be mutated within the scope of the present disclosure. A mutation in a nuclear-encoded photosynthesis gene may be created using any methods appropriate for targeting the disruption of the nuclear gene or may be generated by insertion of a transposable element as described herein. Other methods to disrupt a nuclear encoded gene are known by those skilled in the art, as by use of chemical mutagenesis, radiation, etc. A plant having a mutation in a nuclear-encoded chloroplast protein may be referred to herein as a “mutant plant” or a “mutant line.” Mutant lines as described herein may be produced using the methods of the disclosure and may be used as recipient plant lines for complementation studies as described herein.

In some embodiments, a nuclear mutant line may be maintained in a particular genetic background, in order to maintain plants with the desired genetic mutation. For example, in accordance with the disclosure, a nuclear mutant line as described herein may be maintained in a hybrid genetic background. Plants heterozygous for a genetic mutation as described herein may carry the non-photosynthetic nuclear mutation while exhibiting a photosynthetic phenotype. Plant breeding programs may be used to maintain a desired genetic mutation in a particular plant line or genetic background. Plant breeding programs are well-known in the art and can be modified as necessary depending on the growth requirements of the particular plant species used. For example, in some embodiments, a mutation producing a non-photosynthetic nuclear mutant plant in accordance with the disclosure may be maintained in a hybrid genetic background such as including, but not limited to, an A188×B73 hybrid genetic background. Any genetic background may be used in accordance with the disclosure, as long as the desired mutation is able to be maintained for the desired use.

In some embodiments, a method as described herein may further comprise directly transforming a non-photosynthetic plastid as described herein, for example using a chloroplast transformation vector as described herein. Such a vector may have a functional copy of a gene that was disrupted or mutated such that the functional copy may replace the non-functional gene. In some embodiments, the function of the inactivated gene may be complemented by the presence of the functional copy, for example upon expression of the functional copy of the gene from the chloroplast transformation vector. In other embodiments, the functional copy of the gene may be incorporated into the chloroplast genome and restore function of the gene in that way. In some embodiments, the chloroplast transformation vector may also have a second gene that confers to the plant an agronomically and/or non-agronomically beneficial trait or phenotype as described herein.

In some embodiments, the disclosure also provides a non-photosynthetic chloroplast produced by a method as described herein.

Polynucleotides useful in the present disclosure can be provided in an expression construct. Expression constructs of the disclosure generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements used for expression of nuclear genes include promoters, transcription termination sequences, translation termination sequences, enhancers, and polyadenylation elements. Regulatory elements used for expression of plastid genes include promoters, translational leader sequences, transcription stability and termination sequences and translation termination sequences. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the disclosure can comprise a promoter sequence operably linked to a polynucleotide sequence encoding a polypeptide of the disclosure. Promoters can be incorporated into a polynucleotide using standard techniques known in the art. Multiple copies of promoters or multiple promoters can be used in an expression construct of the disclosure. In certain embodiments, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

If an expression construct as described herein is to be provided in or introduced into a plant cell nucleus, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used. Tissue-specific promoters, for example xylem-specific promoters, such as the promoter of Cald5H, SAD, XCP1, CAD, CesA1, CesA2, CesA3, tubulin gene (TUB) promoter, lipid transfer protein gene (LTP) promoter, or coumarate-4-hydroxylase gene (C4H) promoter (see, for example, Lu et al., 2008; Funk et al., 2002; Sibout et al., 2005; published U.S. application no. 2008/0196125) can be used. Leaf-specific promoters that can be used in a nucleic acid construct of the disclosure include Cab1 promoter (Brusslan and Tobin, 1992), Cab19 promoter (Bassett et al., 2007), PPDK promoter (Matsuoka et al., 1993), and ribulose biphosphate carboxylase (RBCS) promoter (Matsuoka et al. (1994) and U.S. Pat. No. 7,723,575). Other plant leaf-specific promoters that can be used with an expression construct of the disclosure include, but are not limited to, the Act1 promoter (U.S. Published Application No. 20090031441), AS-1 promoter (U.S. Pat. No. 5,256,558), RBC-3A promoter (U.S. Pat. No. 5,023,179), the CaMV 35S promoter (Odell et al., 1985), the enhanced CaMV 35S promoter, the Figwort Mosaic Virus (FMV) promoter (Richins et al., 1987), the mannopine synthase (mas) promoter, the octopine synthase (ocs) promoter, or others such as the promoters from CaMV 19S (Lawton et al., 1987), nos (Ebert et al., 1987), Adh (Walker et al., 1987), sucrose synthase (Yang et al., 1990), α-tubulin, ubiquitin, actin (Wang et al., 1992), cab (Sullivan et al., 1989), PEPCase (Hudspeth et al., 1989) or those associated with the R gene complex (Chandler et al., 1989). See also published U.S. application 2007/006346 and Yamamoto et al. (1997); Kwon et al. (1994); Yamamoto et al. Other promoters that direct expression in the xylem of plants include the 4-coumarate Co-enzyme A ligase (4CL) promoter of Populus described in U.S. Pat. No. 6,831,208. Seed-specific promoters such as the promoter from a β-phaseolin gene (for example, of kidney bean) or a glycinin gene (for example, of soybean), and others, can also be used. Endosperm-specific promoters include, but are not limited to, MEG1 (EPO application No. EP1528104) and those described by Wu et al. (1998), Furtado et al. (2001), and Hwang et al. (2002). Root-specific promoters, such as any of the promoter sequences described in U.S. Pat. No. 6,455,760 or U.S. Pat. No. 6,696,623, or in published U.S. patent application Nos. 20040078841; 20040067506; 20040019934; 20030177536; 20030084486; or 20040123349, can be used with an expression construct of the disclosure. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs of the disclosure.

Methods for identifying and characterizing promoter regions in plant genomic DNA are known in the art and include, for example, those described in the following references: Jordano et al. (1989); Bustos et al. (1989); Green et al. (1988); Meier et al. (1991); and Zhang et al. (1996). U.S. Application Publication No. 2009/0199307 also describes methods for identifying tissue-specific promoters using differential display (see, e.g., U.S. Pat. No. 5,599,672). In differential display, mRNAs are compared from different tissue types. By identifying mRNA species which are present in only a particular tissue type, or set of tissue types, corresponding genes can be identified which are expressed in a tissue specific manner. RNA can be transcribed by reverse transcriptase to produce a cDNA, and the cDNA can be used to isolate clones containing the full-length genes. The cDNA can also be used to isolate homeologous or homologous promoters, enhancers or terminators from the respective gene using, for example, suppression PCR. See also U.S. Pat. No. 5,723,763.

Nuclear Expression constructs of the disclosure may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the disclosure. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences that direct polyadenylation of mRNA transcribed from the expression construct can also be included in the expression construct, and include, but are not limited to, an octopine synthase or nopaline synthase signal.

Polynucleotides of the present disclosure can be composed of either RNA or DNA. In certain embodiments, the polynucleotides are composed of DNA. The subject disclosure also encompasses those polynucleotides that are complementary in sequence to the polynucleotides disclosed herein. Polynucleotides and polypeptides of the disclosure can be provided in purified or isolated form.

Because of the degeneracy of the genetic code, a variety of different polynucleotide sequences can encode polypeptides of the present disclosure. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides of the subject disclosure. These variant or alternative polynucleotide sequences are within the scope of the subject disclosure. As used herein, references to “essentially the same” sequence refers to sequences which encode amino acid substitutions, deletions, additions, or insertions which do not materially alter the functional activity of the polypeptide encoded by the polynucleotides of the present disclosure.

Amino acids can be generally categorized in the following classes: non-polar, uncharged polar, basic, and acidic. Conservative substitutions whereby a polypeptide of the present disclosure having an amino acid of one class is replaced with another amino acid of the same class fall within the scope of the subject disclosure so long as the polypeptide having the substitution still retains substantially the same functional activity as the polypeptide that does not have the substitution. Polynucleotides encoding a polypeptide having one or more amino acid substitutions in the sequence are contemplated within the scope of the present disclosure.

Expression of plastid genes is different from nuclear genes. Plastid gene expression signals include a promoter, translational control region, coding sequence and transcription stability sequence. Plastid transgene expression signals are derived from resident plastid genes, or in some cases, can be derived from bacterial genes or from bacteriophage genes.

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules are capable of forming an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Stringency conditions are known in the art and would be understood by one of skill reading the present disclosure. One of skill in the art will also understand that stringency may be altered as appropriate to ensure optimum results. Complementarity as described herein also refers to the binding of a DNA editing enzyme to its target in vivo or in vitro. One of skill in the art would recognize that variations in complementarity will depend on the particular nucleic acid sequence and will be able to modify conditions as appropriate to account for this.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

Polynucleotides and polypeptides contemplated within the scope of the subject disclosure can also be defined in terms of more particular identity and/or similarity ranges with those sequences of the disclosure specifically exemplified herein. The sequence identity will typically be greater than 60%, greater than 75%, greater than 80%, greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

In some embodiments, the disclosure provides plants generated by a method as described herein. For example, in some embodiments, the disclosure provides plants produced by inactivating a chloroplast-encoded gene as described herein. In other embodiments, the disclosure is also intended to encompass plants produced by inactivating a nuclear-encoded gene that controls a chloroplast-encoded gene as described herein. In such cases, the nuclear-encoded gene may encode a transcription factor or a translation factor that serves to control a gene encoded by the chloroplast and required for photosynthesis. Plants may be produced using any methods known in the art, and using any appropriate growth conditions and/or reagents. One of skill in the art will understand and be able to perform plant growth techniques and methods. In some embodiments, callus may be generated from a non-photosynthetic mutant plant as described herein using standard protocols known in the art. As described herein, a non-photosynthetic mutant plant of the disclosure is intended to encompass a plant in which a chloroplast-encoded gene is inactivated or disrupted, as well as a plant in which a nuclear-encoded gene is inactivated or disrupted that controls a chloroplast gene required for photosynthesis.

As described herein, callus tissue may be generated from a non-photosynthetic mutant plant or plant parts. As would be understood by one of skill in the art, callus may be generated from any type of plant tissue, for example, embryo tissue, seeds, such as seed explant tissue, stem, meristem, leaf, root, etc. Callus generated from such plants and grown in culture may then be used as recipients for complementation experiments as described herein. Other embodiments provide a plant part of a plant as described herein, selected from the group consisting of embryo, seed, stem, callus, meristem, leaf, root, or any plant part from which viable cells may be obtained and used in culture. In another embodiment, the disclosure provides a seed produced by a plant as described herein. Some embodiments provide for a plant regenerated from a callus, or any part of a plant resulting from a mutant, non-photosynthetic plant as described herein. Other embodiments provide a plant part of a plant as described herein, selected from the group consisting of a seed, stem, callus, meristem, leaf, root, or the like. In another embodiment, the disclosure provides a seed produced by a plant as described herein.

Nuclear transformation in monocots typically requires embryogenic callus or scutellar tissue from freshly isolated immature embryos, as these are the most highly regenerable tissues in monocot cell culture. Recently, it has been shown that over-expression of maize Baby boom (Bbm) and Wuschel (Wus) genes can rapidly initiate embryogenic callus growth from a variety of monocot tissues, leading to stable nuclear transformation from tissues such as mature seed or leaf segments. Furthermore, Bbm and Wus gene over-expression can enable nuclear transformation in otherwise recalcitrant genotypes (Lowe et al. 2016). Non-photosynthetic mutants are anticipated to grow as embryogenic callus on media containing sugars. It is anticipated that over-expression of Bbm and Wus genes can also enhance the growth of these non-photosynthetic mutants or enhance selection for greening and photosynthetic competence in embryogenic callus and other tissues such as leaf. In certain embodiments, Bbm and Wus polypeptides and genes set forth in US Patent Appl. Pub. No. 20170342431; and U.S. Pat. No. 7,256,322, both incorporated herein by reference in their entirety, can be adapted for use in the methods provided herein to provide for plastid transformation and regeneration of transplastomic plants. Additional morphogenic genes have also recently been shown to enable embryogenesis in both monocot and dicot plant species. For example, ZmGRF5-like1 and 2 increased transgenic embryogenic callus formation (A188) and increased proliferation of callus (Kong et al 2020) whereas overexpression of the WOX5 gene drastically promoted de novo shoot regeneration from callus (Lee et al. 2022). Likewise, GRF4-GIF1 overexpression increased the frequency of regeneration of transgenic wheat embryos from callus by 8-fold (Debernardi et al 2020). Similarly, in dicots, overexpression of AtGRF5, BnGRF5-like, AtGRF6 or AtGRF9 resulted in increased transgenic sectors in developing callus and in soybean more meristem initials were observed at axillary nodes from overexpression of GFR5 and GmGRF5-like (Kong et al 2020). While demonstration of de novo shoot regeneration from other tissues yet needs to be shown, these examples indicates that purification of homoplasmic chloroplast lines can be purified from various tissues in both monocots and dicots using several different morphogenic gene options.

In some embodiments, the plants of the present disclosure may also further exhibit one or more agronomically and/or non-agronomically beneficial traits. As used herein, an “agronomically beneficial trait” refers to a trait or characteristic that is desirable or important for a particular crop or species. Agronomically beneficial traits may result in increased commercial value of the plant or crop, such as by providing improved taste, resistance to herbicides or pests, environmental tolerance, or may provide a selective advantage for the plant. Such traits may be desirable for, for example, a seed company, a grower, or a grain processor. Agronomically beneficial traits include traits effected by genes that alter quality traits such as oil (e.g.; fatty acid), protein, amino acid, starch, and/or other nutrient content and/or profiles in plant products including seed and seed meal. Agronomically beneficial traits include traits effected by genes, that provide for improved processing or use of plant products including seed meal, defatted seed meal, and the like as food or animal feed products. Examples of agronomically beneficial traits may include any desired characteristic, such as including, but not limited to, herbicide resistance (e.g., tolerance to glyphosate, glufosinate, dicamba or other auxin analogs), virus resistance, bacterial pathogen resistance, insect resistance, nematode resistance, and fungal resistance resistance (e.g., biotic-stress tolerance traits). Such a trait may also be one that increases plant vigor or yield, including traits that allow a plant to grow at different temperatures, soil conditions, and levels of sunlight and precipitation, resistance (e.g., abiotic-stress tolerance traits) or one that allows identification of a plant exhibiting a trait of interest (e.g., selectable marker gene, flower color or pattern, seed coat color, etc.). Various traits of interest, as well as methods for introducing these traits into a plant, are known in the art. For example, herbicide resistant results from over-expression of EPSPS, HPPD, bar, or AHAS herbicide resistant genes, cry1A, cry2A, or cry3A crystal proteins derived from Bacillus thuringiensis to provide insect resistance, defensins, or anti-fungal peptides to provide resistance to bacterial and fungal species. Non-agronomically beneficial traits include genes that encode proteins or enzymes that can be used in pharmaceutical or industrial applications.

Methods of plant transformation are well-known in the art. Techniques for transforming plant cells with a gene include, for example, Agrobacterium infection, biolistic methods, electroporation, DNA coated particles, calcium chloride treatment, PEG-mediated transformation, etc. (see, e.g., Nagel et al., 1990; Song et al., 2006; de la Pena et al., 1987; and Klein et al., 1993). U.S. Pat. No. 5,661,017 teaches methods and materials for transforming an algal cell with a heterologous polynucleotide. Suitable methods for transformation of host cells for use with the current disclosure are believed to include virtually any method by which DNA can be introduced into a cell (see, e.g., Miki et al., 1993), for example by Agrobacterium-mediated transformation (U.S. Pat. Nos. 5,563,055; 5,591,616; 5,693,512; 5,824,877; 5,981,840; 6,384,301; Gelvin, 2003; and Broothaerts et al., 2005) and by acceleration of DNA coated particles (U.S. Pat. Nos. 5,015,580; 5,550,318; 5,538,880; 6,160,208; 6,399,861; and 6,403,865), etc. Through the application of techniques such as these, the cells of virtually any species may be stably transformed.

In some embodiments, plant transformation can be achieved by biolistic transformation, or bombardment. As used herein, bombardment refers to a method of insertion of genetic material into a cell wherein the genetic material is forcibly injected into the cell with the use of a biolistic gun or related device or vehicle. Using biolistic transformation, a plasmid carrying a wild-type chloroplast genome segment as described herein may be delivered to a cell, such that integration of the transforming DNA complements the deletion mutant and restores the ability of the cell to perform photosynthesis. Photosynthetic capability may be verified by the ability of the cell or callus to grow on selective media. Selective media are widely known and available in the art. Media for cell culture may be altered as appropriate for the particular application without altering the scope of the disclosure as described herein.

Following transformation, cells can be selected, re-differentiated or regenerated, and grown as callus or grown into plants that contain and express a polynucleotide of the disclosure using standard methods known in the art. The seeds and other plant tissue and progeny of any transformed or transgenic plant cells or plants of the disclosure are also included within the scope of the present disclosure.

Various methods for selecting transformed cells have been described. For example, one might utilize a drug resistance marker such as a neomycin phosphotransferase protein to confer resistance to kanamycin or to use 5-enolpyruvyl shikimate phosphate synthase to confer tolerance to glyphosate. In another embodiment, a carotenoid synthase is used to create an orange pigment that can be visually identified. These three exemplary approaches can each be used effectively to isolate a cell or multicellular organism or tissue thereof that has been transformed and/or modified as described herein. In some embodiments, the present methods may eliminate the need for a selection step with the generation of mutant non-photosynthetic plants as described herein.

Numerous permutations of methods for biolistics for plant transformation are known and available in the art. For example, in some embodiments, bombardment and selection experiments of dark-grown callus may be performed as described herein by bombardment of dark-grown callus, followed by selection in the light, on media requiring photosynthesis for growth, until green callus (putative transformed lines) is observed. In other embodiments, bombardment of dark-grown callus shifted into the light for several days until such time that greening of wild-type callus would normally occur, followed by selection in the light, on media requiring photosynthesis for growth, until green callus (putative transformed lines) is observed. In some embodiments, bombardment of dark-grown callus may be followed by immediate plant regeneration on media requiring photosynthesis for growth, and any resulting green regenerated plants may be evaluated as having been complemented.

In some embodiments, a non-naturally occurring sequence-specific or sequence-directed exogenous nucleic acid is introduced into a cell in order to introduce a mutation in a gene required for photosynthesis in the cell, or in an organism comprised of such cells. In some embodiments, the cell is a plant cell and the mutation results in the inability of the cell to undergo photosynthesis. The ability to generate such a cell, or an organism derived therefrom depends on introducing an exogenous nucleic acid into the cell using, for example, transformation vectors and cassettes described herein.

A polypeptide useful in accordance with the disclosure may be isolated, non-naturally occurring, recombinant, or engineered nucleic acid-binding proteins that have nucleic acid or DNA binding regions containing polypeptide monomer repeats designed to target specific nucleic acid sequences.

In some embodiments, an exogenous nucleic acid as described herein may be transiently introduced into the cell. In certain embodiments, the introduced exogenous nucleic acid is provided in sufficient quantity to modify the cell but does not persist after a contemplated period of time has passed or after one or more cell divisions. In such embodiments, no further steps are needed to remove or segregate the exogenous nucleic acid from the modified cell.

In another embodiment, mRNA encoding the exogenous nucleic acid is introduced into a cell. In such embodiments, the mRNA is translated to produce the exogenous nucleic acid in sufficient quantity to modify the cell but does not persist after a contemplated period of time has passed or after one or more cell divisions. In such embodiments, no further steps are needed to remove or segregate the exogenous nucleic acid from the modified cell.

In one embodiment of this disclosure, a catalytically active exogenous nucleic acid is prepared in vitro prior to introduction to a cell, including a prokaryotic or eukaryotic cell. The method of preparing a exogenous nucleic acid depends on its type and properties and would be known by one of skill in the art. After expression, the exogenous nucleic acid is isolated, refolded if needed, purified and optionally treated to remove any purification tags, such as a His-tag. Once crude, partially purified, or more completely purified exogenous nucleic acid are obtained, it may be introduced to, for example, a plant cell via electroporation, by bombardment with coated particles, by chemical transfection or by some other means of transport across a cell membrane as described herein. Methods for introducing nucleic acids into bacterial and animal cells are similarly well known in the art. In the case of Agrobacterium-mediated plant transformation methods, the exogenous nucleic acid can be expressed in Agrobacterium as a recombinant protein, fused to an appropriate domain of a Vir protein such that it is transported to the plant cell (Vergunst et al., 2000). The protein can also be delivered using nanoparticles, which can deliver a combination of active protein and nucleic acid (Torney et al., 2007). Once a sufficient quantity of the exogenous nucleic acid is introduced so that an effective amount is present, the target site or sites are looped out. It is also recognized that one skilled in the art might create an exogenous nucleic acid that is inactive but is activated in vivo by native processing machinery.

In another embodiment, a construct that will transiently express a exogenous nucleic acid is created and introduced into a cell. In yet another embodiment, the vector will produce sufficient quantities of the exogenous nucleic acid in order for the desired target site or sites to be effectively recombined. For instance, the disclosure contemplates preparation of a vector that can be bombarded, electroporated, chemically transfected or transported by some other means across the plant cell membrane. Such a vector could have several useful properties. For instance, in one embodiment, the vector can replicate in a bacterial host such that the vector can be produced and purified in sufficient quantities for a transient expression. In another embodiment, the vector can encode a drug resistance gene to allow selection for the vector in a host, or the vector can also comprise an expression cassette to provide for the expression of the exogenous nucleic acid in an organism. In a further embodiment, the expression cassette could contain a promoter region, a 5′ untranslated region, an optional intron to aid expression, a multiple cloning site to allow facile introduction of a sequence encoding an exogenous nucleic acid, and a 3′ UTR. In some embodiments, it can be beneficial to include unique restriction sites at one or at each end of the expression cassette to allow the production and isolation of a linear expression cassette, which can then be free of other vector elements. The untranslated leader regions, in certain embodiments, can be plant-derived untranslated regions. Use of an intron, which can be plant-derived, is contemplated when the expression cassette is being transformed or transfected into a monocot cell.

As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. As used herein, an expression cassette may comprise an exogenous nucleic acid operably linked to a promoter as described herein and a chloroplast transit peptide.

In one exemplary approach, a transient expression vector may be introduced into a cell using a bacterial or viral vector host. For example, Agrobacterium is one such bacterial vector that can be used to introduce a transient expression vector into a host cell. When using a bacterial, viral or other vector host system, the transient expression vector is contained within the host vector system. For example, if the Agrobacterium host system is used, the transient expression cassette would be flanked by one or more T-DNA borders and cloned into a binary vector. Many such vector systems have been identified in the art (reviewed in Hellens et al., 2000). In embodiments whereby the exogenous nucleic acid is transiently introduced in sufficient quantities to modify a cell, a method of selecting the modified cell may be employed. In the present case, one may look for non-green plant sectors or non-green embryo tissues or callus derived from embryos. In one such method, a second nucleic acid molecule containing a selectable marker may be co-introduced with the transient exogenous nucleic acid.

Cell transformation systems have been described in the art and descriptions include a variety of transformation vectors. For example, for plant transformations, two principal methods include Agrobacterium-mediated transformation and particle gun bombardment-mediated transformation. In both cases, the exogenous nucleic acid is introduced via an expression cassette. The cassette may contain one or more of the following elements: a promoter element that can be used to express the exogenous nucleic acid; a 5′ untranslated region to enhance expression; an intron element to further enhance expression in certain cell types, such as monocot cells; a multiple-cloning site to provide convenient restriction sites for inserting the exogenous nucleic acid-encoding sequence and other desired elements; and a 3′ untranslated region to provide for efficient termination of the expressed transcript. For particle bombardment or with protoplast transformation, the expression cassette can be an isolated linear fragment or may be part of a larger construct that might contain bacterial replication elements, bacterial selectable markers or other elements. The exogenous nucleic acid expression cassette may be physically linked to a marker cassette or may be mixed with a second nucleic acid molecule encoding a marker cassette. The marker cassette is comprised of necessary elements to express a visual or selectable marker that allows for efficient selection of transformed cells. In the case of Agrobacterium-mediated transformation, the expression cassette may be adjacent to or between flanking T-DNA borders and contained within a binary vector. In another embodiment, the expression cassette may be outside of the T-DNA. The presence of the expression cassette in a cell may be manipulated by positive or negative selection regime(s). Furthermore, a selectable marker cassette may also be within or adjacent to the same T-DNA borders or may be somewhere else within a second T-DNA on the binary vector (e.g., a 2 T-DNA system).

In another embodiment, cells that have been modified by an exogenous nucleic acid, either transiently or stably, are carried forward along with unmodified cells. The cells can be sub-divided into independent clonally derived lines or can be used to regenerate independently derived organisms. Individual plants or animals or clonal populations regenerated from such cells can be used to generate independently derived lines. At any of these stages a molecular assay can be employed to screen for cells, organisms or lines that have been modified. Cells, organisms or lines that have been modified continue to be propagated and unmodified cells, organisms or lines are discarded. In these embodiments, the presence of an active exogenous nucleic acid in a cell is essential to ensure the efficiency of the overall process.

### Expression Strategies

Promoters for transformation have been described in the art; thus, the disclosure provides, in certain embodiments, novel combinations of promoters and a sequence encoding an exogenous nucleic acid, to allow for specifically introducing a recombination event into endogenous DNA (i.e., a genome). In one embodiment, a constitutive promoter is cloned 5′ to a sequence encoding an exogenous nucleic acid, in order to constitutively express the exogenous nucleic acid in transformed cells. This may be desirable when the activity of the exogenous nucleic acid is low or the frequency of finding and recombining the target site is low. It may also be desirable when a promoter for a specific cell type, such as the germ line, is not known for a given species of interest.

In another embodiment, an inducible promoter can be used to turn on expression of the exogenous nucleic acid under certain conditions. For example, a cold shock promoter cloned upstream of an exogenous nucleic acid might be used to induce the exogenous nucleic acid under cold temperatures. Other environmentally inducible promoters have been described and can be used in a novel combination with an exogenous nucleic acid-encoding sequence. Another type of inducible promoter is a chemically inducible promoter. Such promoters can be precisely activated by the application of a chemical inducer. Examples of chemical inducible promoters include the steroid inducible promoter and a quorum sensing promoter (see, e.g., You et al., 2006; U.S. Patent Application Publication No. 2005/0227285). Recently it has been shown that modified RNA molecules comprising a ligand specific aptamer and riboswitch can be used to chemically regulate the expression of a target gene (Tucker et al, 2005; International Publication No. WO2006073727). Such a riboregulator can be used to control the expression of an exogenous nucleic acid-encoding gene by the addition or elimination of a chemical ligand.

In other embodiments, the promoter may be a tissue specific promoter, a developmentally regulated promoter, or a cell cycle regulated promoter. Certain contemplated promoters include ones that only express in the germline or reproductive cells, among others. Such developmentally regulated promoters have the advantage of limiting the expression of the exogenous nucleic acid to only those cells in which DNA is inherited in subsequent generations. Therefore, a genetic modification by an exogenous nucleic acid (i.e., genetic recombination) is limited only to cells that are involved in transmitting their genome from one generation to the next. This might be useful if broader expression of the exogenous nucleic acid were genotoxic or had other unwanted effects.

Another contemplated promoter is a promoter that directs developmentally regulated expression limited to reproductive cells just before or during meiosis. Such a promoter has the advantage of expressing the exogenous nucleic acid only in cells that have the potential to pass on their genome to a subsequent generation. Examples of such promoters include the promoters of genes encoding DNA ligases, recombinases, and replicases, among others.

In addition to promoters, this disclosure provides for 5′ untranslated regions, introns and 3′ untranslated regions that can be uniquely combined with a exogenous nucleic acid-encoding sequence to create novel expression cassettes with utility for genome engineering.

The disclosure also provides molecular assays for detecting and characterizing cells that have been modified as described herein. These assays include but are not limited to genotyping reactions, a PCR assay, a sequencing reaction or other molecular assay. Design and synthesis of nucleic acid primers useful for such assays, for instance to assay for the occurrence of a recombination event, are also contemplated.

Genotyping of cells may be performed on any cells or tissue as appropriate with the disclosure, including callus cells or tissue. The genotype of callus derived from transformed plant embryos can be determined by, for example, PCR analysis, using PCR amplification of the PPR10 gene.

Chloroplasts, among other plastids, are believed to have originated from bacteria and as such have retained some of the bacterial gene expression characteristics. For example, chloroplasts of land plants have polycistronic transcription units that resemble bacterial operons. In addition, chloroplast ribosomes are similar in protein content and antibiotic sensitivities to bacterial ribosomes. Chloroplasts also have a bacterial-type RNA polymerase for chloroplast transcription, and ribonucleases that are derived from those in bacteria are involved in chloroplast RNA turnover.

Chloroplasts also exhibit similarities to eukaryotic gene expression, including the presence of introns, a phage-type RNA polymerase, modification of mRNA sequences by RNA editing, and processing of polycistronic primary transcripts to generate complex transcript populations.

Plastids regulate protein accumulation via translational control. For photosynthetic genes, for example, transcription is constitutive but the protein product is translated only in green tissues in the light. Translational control sequences can derive the same plastid gene as the promoter or a different plastid gene to create a chimeric promoter/leader construct that combines ideal functions. For example, a strong promoter may be combined with a leader sequence that directs translation across both light- and dark-grown tissues. An example of such a leader sequence is derived from the plastid clpP gene (Zhang 2012)). A bacterial-derived translational control sequence, such as in the bacteriophage gene 10 leader sequence (G10L), can also be used to direct high-level constitutive expression of multiple transgenes in plastids (Ye et al., 2001)). For transcript stability, a 3′-UTR region is used. The 3′UTR terminates transcription via a stem/loop region that forms in the RNA independent of the genetic background. Therefore, a 3′UTR region from a homologous plastid gene, heterologous plastid gene or bacteria may be used. Plastid expression signals that are derived from a different plant species may have an advantage in that the reduced nucleotide sequence identity may reduce or eliminate the possibility of intragenic recombination.

In some embodiments, a selectable marker gene and gene(s)-of-interest as described herein may be expressed from gene expression elements that function in plant plastids. As most plastid genes are constitutively expressed, a plastid promoter may be chosen based on its relative strength. In most cases, plastid promoter sequences derive from the plastid genome of the same plant species. Plastids contain different promoter types; those recognized by the plastid-encoded RNA polymerase (PEP), the nuclear-encoded RNA polymerase (NEP) or both. PEP promoter elements resemble the bacterial-like-10 and -35 recognition elements whereas NEP promoters have a single core promoter element. Plastid genes with NEP promoter typically are over-expressed in undeveloped plastid types, while plastid genes with PEP promoter elements are typically over-expressed in developed chloroplasts. Plastid genes with both PEP and NEP promoters are highly transcribed in both tissue types. An example of a NEP promoter active in non-green tissues is derived from the clpP gene. A strong constitutive promoter with both NEP and PEP elements is derived from the 16SrDNA gene (Prrn).

Evaluation of non-photosynthetic mutants in species such as maize, Arabidopsis, and Chlamydomonas has demonstrated that a number of nuclear-encoded RNA-binding proteins participate in the expression of chloroplast genes. These can serve as transcription factors or translation factors. Thus, in some embodiments, a nuclear-encoded gene as described herein may function in the cell to control expression of a chloroplast photosynthesis gene. For example, a nuclear-encoded gene may encode a transcription factor for a chloroplast photosynthesis gene, or may encode a translation factor for a chloroplast photosynthesis gene. In other embodiments, a nuclear-encoded gene may encode a protein functional as a structural protein in the chloroplast. In accordance with the disclosure, a nuclear-encoded gene that results in a non-photosynthetic mutant plant may serve any function in the process of photosynthesis such that its inactivation or mutation eliminates the cell's ability to perform photosynthesis. One of skill in the art will be able to identify appropriate genes for this purpose, which are intended to fall within the scope of the present disclosure.

The disclosure further provides a kit comprising a single-use container comprising a callus or seed produced from a plant part as described herein. In some embodiments, it may be desirable to provide a plant part and reagents for producing callus tissue. In such a case, sterile reagents and tissue may be provided as appropriate. A kit may further comprise reagents for cell transformation, cell culture, or both.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, cell transformation, tissue culture, genetic complementation assays, or any assay useful in accordance with the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, media components, such as salts including MgCl₂, a polymerase enzyme, and deoxyribonucleotides, and the like, reagents for DNA isolation, or the like, as described herein. Such reagents or components are well known in the art. Where appropriate, reagents included with such a kit may be provided either in the same container or media as a primer pair or multiple primer pairs, or may alternatively be placed in a second or additional distinct container into which an additional composition or reagents may be placed and suitably aliquoted. Alternatively, reagents may be provided in a single container means. A kit of the disclosure may also include instructions for use, including storage requirements for individual components as appropriate.

### IX. Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR § 1.822 is used.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, “homoplasmic” refers to a eukaryotic plant cell whose copies of plastid DNA are all identical. Homoplasmic plastid DNA copies may be normal or mutated. In the case; however, that a mixed population of plastid DNA molecules exist, these are termed heteroplasmic, i.e., only occurring in some copies of plastid DNA. Heteroplasmic plastid DNA could arise, for example, when some copies of plastid DNA are mutated and some copies of plastid DNA are wild-type. Homoplasmy may occur naturally or otherwise.

As used herein, “non-photosynthetic” refers to a plant that is incapable of performing photosynthesis. Non-photosynthetic plants may be the result of a genetic mutation, whether natural or induced. A non-photosynthetic plant of the disclosure may be the result of a mutation in a chloroplast photosynthesis gene, or a nuclear gene that is involved with photosynthesis. One or more genes may be involved.

As used herein, “callus” refers to growing mass of unorganized plant cells. Callus culture is known in the art, and formation of callus tissue may be performed under sterile tissue culture conditions using reagents as appropriate for the particular application. Type I callus (less differentiated) and Type II callus (more differentiated) may grow on different types of media and at different rates. Individual embryos were placed onto medium in Petri plates in a grid pattern and grown in the dark at 28° C.

As used herein, “domain” refers to a polypeptide that includes an amino acid sequence of an entire polypeptide or a functional portion of a polypeptide. Certain functional subsequences are known, and if they are not known, can be determined by truncating a known sequence and determining whether the truncated sequence yields a functional polypeptide.

As used herein, “expression construct” refers to a DNA construct that includes an encoded exogenous nucleic acid protein that can be transcribed.

As used herein, “exogenous DNA sequence” refers to a DNA sequence that originates outside the host cell. Such a DNA sequence can be obtained from a different species, or the same species, as that of the cell into which it is being delivered.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any article (e.g., plant, plant part such as a seed, or plant cell; gene or protein), composition, or device that “comprises,” “has” or “includes” one or more examples or features is not limited to possessing only those one or more examples or features and can cover other unlisted examples or features.

All methods described herein can be performed in any suitable order unless otherwise indicated herein or otherwise clearly contradicted by context. In addition, embodiments described herein in reference to complementation of chloroplast-encoded genes may also be appropriate for complementation of mutated nuclear genes encoding chloroplast-localized proteins involved in photosynthesis, and are therefore included in all embodiments as appropriate. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Embodiments

1. A method of expressing an agronomically beneficial trait in a plant plastid comprising: (a) utilizing a non-photosynthetic plant or plant part homozygous for the loss of function of a nuclear-encoded gene involved in photosynthesis in the chloroplast; (b) growing a callus of the non-photosynthetic plant in culture; (c) transforming mutant plant-line callus with a plastid transformation vector comprising a copy of the nuclear-encoded gene functional in plant plastids and an agronomically beneficial trait gene; and (d) selecting green, photosynthetic callus.
2. The method of embodiment 1, wherein the plastid transformation vector comprises a chloroplast transformation vector.
3. The method of embodiment 1, wherein the selection step is performed in light growth conditions.
4. The method of embodiment 1, wherein the plant comprises a corn plant or a soy plant.
5. The method of embodiment 1, wherein the recipient mutant plant line is a non-photosynthetic nuclear mutant plant line.
6. The method of embodiment 5, wherein the mutant corn plant is a ppr10 mutant line, an atpc mutant or chli mutant line.
7. The method of embodiment 6, wherein the corn ppr mutant line is ppr10-1 or ppr10-2.
8. The method of embodiment 5, wherein the mutant soy plant is a chli mutant line or a psbP mutant line.
9. The method of embodiment 1, wherein the corn mutant line is maintained in a hybrid genetic background.
10. The method of embodiment 9, wherein the corn hybrid genetic background is A188×B73.
11. A plant produced by the method of embodiment 1.
12. A plant part of the plant of embodiment 11, selected from the group consisting of a seed, embryo, stem, callus, meristem, leaf, and root.
13. A seed produced by the plant of embodiment 11.
14. A method of expressing an agronomically beneficial trait in a plant comprising: (a) selecting a recipient mutant plant or callus homozygous for the loss of function of a nuclear-encoded gene involved in photosynthesis in the chloroplast; (b) growing a callus of the selected recipient mutant plant in culture; (c) transforming the mutant plant-line callus with a nuclear transformation vector comprising a wild-type copy of the nuclear-encoded gene and one or more agronomically beneficial trait genes; (d) selecting green, photosynthetic callus in the light; and (e) regenerating a green plant and the agronomically beneficial trait.
15. A kit comprising: a single-use container comprising a callus or seed produced from the plant part of embodiment 12.
16. The kit of embodiment 15, further comprising reagents for transformation, cell culture, or both.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1. Plastid Transformation of Ppr10, Atpc or Chli Photosynthetic Mutant Recipient Lines and Selection for Photosynthetic Competence in the Light

Homozygous ppr10 mutant lines were used as recipient lines for plastid transformation. These lines were maintained as dark-grown callus.

### Growth of Ppr10 Plants and Establishment of Embryogenic Callus

Seeds from heterozygous plants carrying nuclear ppr10-1 or ppr10-2 mutant alleles (described in Pfalz et al, 2009) were obtained. Ppr10-1 and ppr10-2 mutations were derived from a Mu transposon insertion into different regions of the PPR10 gene. Ppr10 mutant lines were maintained as heterozygotes in an A188×B73 hybrid genetic background or any other suitable genetic background. Sequences of the PPR10 protein and cDNA are set forth in SEQ ID NO: 27 and SEQ ID NO:28.

Examples of other nuclear-encoded genes that when mutated may generate a similar pale-green or albino phenotype are provided in Table 1.

Seeds from heterozygous plants carrying ppr10-1 or ppr10-2 alleles were sown in soil in the greenhouse in 2.5″ Nu Pots. Each seed was planted in a custom blend of MetroMix 360 and Turface, a 3:1 (MM360:T) blend by volume. Plants were grown in a greenhouse with a 16 hr/8 hr day/night cycle with 28° C./22° C. day/night temps. The seeds were watered with clear reverse osmosis (RO) water until the V3 stage, and with 15-5-15 CaMg fertilizer after V3.

Plants were grown to maturity in the greenhouse. When silks emerged, plants were checked for pollen. If adequate pollen was shed, the husk of the ear was cut to 1 inch above the tip of the cob and the tassel bagged in the afternoon. The next morning, silk was expected to have pushed 0.5 to 1 inch through the ear husk. The plant was bent over and the tassel bag hit with the hand repeatedly for 10 to 15 seconds to dislodge the pollen from the tassel. The bag was removed so that pollen was not lost and the pollen was then poured onto the silk. The pollination bag was then placed on the ear.

At ˜9-11 days after pollen fertilization, the ear was harvested from the plant. At this time, immature embryos were ˜1.5-2 mm and were placed on callus induction medium MSW57 or N6 in the dark. Type I callus (less differentiated) formed on MSW57 medium and Type II (more differentiated) embryogenic callus formed on N6 medium within ˜2-3 weeks. Individual embryos were placed onto medium in Petri plates in a grid pattern and grown in the dark at 28° C.

Individual embryos derived from ppr10 heterozygous mutant lines segregated as wild-type (PPR10/PPR10; 25%), heterozygous (PPR10/ppr10; 50%), or homozygous mutant (ppr10/ppr10; 25%) genotypes. The genotype of callus derived from these segregating embryos could be determined by PCR analysis, using PCR amplification of the PPR10 gene or a mixture of PCR primers that were designed to amplify the wild-type PPR10 gene that was disrupted with the Mu transposon.

Growth of Ppr10 Plants and Establishment of Callus from Nodal Sections

Seeds derived from ppr10-1 and ppr10-2 heterozygous mutant lines were sown in sterile tissue culture on MSV S34 medium (Table 2).

TABLE 2
Media Components for Growth of Mutant Plant Lines

| | Amount/L | | Amount/L | | Amount/L | | Amount/L | | Amount/L | |
|---|---|---|---|---|---|---|---|---|---|---|
| Components (stock conc.) | MSV S34 | | MSW57 | | DBC3 | | DBC3-2 | | MS3 | |
| MS salts | 4.4 | g | 4.4 | g | 4.4 | g | 4.4 | g | 4.4 | g |
| MS vitamin 1000x | 1 | mL | 1 | mL | | | | | | |
| ThiamineHCl (1 mg/ml) | — | | 0.5 | mL | 1 | mL | 1 | mL | 1 | mL |
| Maltose | 40 | g | — | | 30 | g | 30 | g | 40 | g |
| Casein Hydrolysate | 0.1 | g | — | | 1 | g | 1 | g | 0.5 | g |
| Casamino Acids | — | | 0.5 | g | | | | | | |
| MES | 1.95 | g | — | | — | | — | | 1.95 | g |
| Magnesium Chloride | 0.75 | g | — | | — | | — | | | |
| Sucrose | — | | 30 | g | | | | | | |
| myo-Innositol | | | | | 0.25 | g | 0.25 | g | | |
| Copper sulfate (0.05M) | | | | | 98 | μL | 98 | μL | | |
| Glutamine | 0.5 | g | — | | — | | — | | | |
| L-Proline | — | | 1.38 | g | 0.69 | g | 0.69 | g | | |
| Post Autoclave additives | | | | | | | | | | |
| 2,4-D (1 mg/ml) | — | | 0.5 | mL | 1 | mL | 0.5 | mL | | |
| Pictoram (1 mg/ml) | 10 | mL | 2.2 | mL | | | | | 2.2 | mL |
| BAP (1 mg/ml) | 3 | mL | — | | 0.5 | mL | 0.5 | mL | 1 | mL |
| Ascorbic Acid (50 mg/ml) | 2 | mL | — | | — | | — | | 2 | mL |
| Silver Nitrate (2 mg/ml) | — | | 1.7 | mL | | | | | | |

| Component | N6 | |
|---|---|---|
| N6 salts | 4 | g/L |
| Sucrose | 20 | g/L |
| 1-Proline | 2.88 | g/L |
| Casamino acids | 100 | mg/L |
| Glycine* | 2 | mg/L |
| Nicotinic Acid* | 0.5 | mg/L |
| Pyridoxine HCl* | 0.5 | mg/L |
| Thiamine HCl* | 1 | mg/L |
| Post Autoclave additives | | |
| 2,4-D | 1 | mg/L |
| Silver Nitrate | 10 | uM |
| Phytagel | 2 | g/L |

After ˜7-10 days, seedlings that were homozygous for ppr10 mutation could be distinguished by their yellow or albino appearance. Nodal sections containing the meristem were dissected from ˜7-10-day-old seedlings, cut longitudinally, and placed cut side down onto MSW57 medium in the light to form callus as described in U.S. Pat. No. 9,267,144, incorporated herein by reference. The remaining plant material was used for PCR analysis to confirm the genotypes of the embryos for validation of homozygous mutant seedlings using PCR amplification of the PPR10 gene or a mixture of PCR primers that were designed to amplify the wild-type PPR10 gene that was disrupted with the Mu transposon. Nodal sections derived from homozygous mutant seedlings were used for subsequent callus formation. Since seedlings were germinated in aseptic conditions, there was no need for surface sterilization, and nodal sections could be placed directly into sterile tissue culture.

In some cases, seed derived from ppr10 heterozygous mutant lines were sown in soil in the greenhouse. In this case, homozygous mutant seedlings were identified as albino seedlings and could survive in soil for about 1-2 weeks. Seedlings (above-ground portion) were harvested and surface-sterilized in 40% bleach solution for 20 minutes. After extensive washing with sterile water, nodal sections were dissected as above and placed into sterile tissue culture on MSV57 medium in the dark or the light.

Callus tissue was routinely sub-cultured on N6 or MSW57 medium in the dark to amplify embryogenic callus for subsequent experiments. Growth of homozygous mutants was slow, presumably due to pleiotropic effects of the mutant, and may be enhanced by addition of 5 g/L or 10 g/L glucose to the medium.

PCR analysis to identify homozygous ppr10 mutant callus was performed using the following primers:

PPR10F:
(SEQ ID NO: 16)
5′-GACATCACGTCCCTGCTCAA
PPR10R:
(SEQ ID NO: 17)
5′-TTCCCAACATTCCCATAGGC
mu1:
(SEQ ID NO: 18)
5′-GCCTCCATTTCGTCGAATCCC

The PPR10 gene was amplified using a mixture of PPR10F and PPR10R PCR primers. The PPR10 gene that was disrupted by a Mu transposon was detected using a mixture of PPR10F and mu1 primers.

### Selection of Plastid Transformants by Complementation of the Dark-Grown Non-Photosynthetic Recipient

Callus derived from homozygous ppr10 mutant embryos was maintained and amplified in the dark to be used as recipient for all subsequent plastid transformation experiments. Dark-grown mutant callus was non-green. It was expected that dark-grown non-photosynthetic mutant callus would remain non-green when shifted into the light, due to the lack of chlorophyll accumulation expected in a non-photosynthetic mutant line. However, upon shifting of dark-grown ppr10 mutant callus to the light on MSW57 medium or another suitable medium such as DBC3 or DBC3-2, some of the mutant callus began to turn green on medium containing standard 3% sucrose or maltose. Partial greening on sucrose medium may be because high sugar content can partially rescue the non-photosynthetic phenotype.

An experiment was designed to identify a medium with reduced sugar content that would prevent ppr10 mutant callus from becoming green in the light while still allowing wild-type callus to become and remain fully green under the same conditions.

The tables below outlines testing of mutant (2 independent callus lines, #53 and #62) and wild-type callus for growth and greening response on DBC3 medium with differing amounts of maltose or maltose with sucrose.

TABLE 3
Testing of Mutant and Wild-Type Callus for Growth and Greening

| Experiment 3. | All observations recorded at 28 days of treatment | | |
|---|---|---|---|
| Treatment | Line 53 | Line 62 | Wildtype |
| DBC3 control (30 g/L Maltose) | 20-30% greening, 100% growth | 20-25% greening, 70% growth | 60% greening, 100% growth |
| DBC3 (15 g/L Maltose) | 10-15% greening, 65% growth | 10-15% greening, 55% growth | 30% greening, 85% growth |
| DBC3 (10 g/L Maltose) | 5-10% greening, 55% growth | 5-10% greening, 20% growth | 20% greening, 70% growth |
| DBC3 (5 g/L Maltose) | 5-10% greening, 35% growth | 5% greening, 40% growth | 10-15% greening, 40% growth |
| DBC3 (10 g/L Maltose + 5 g/L Sucrose) | 20-25% greening, 80% growth | 10-15% greening, 70% growth | 30-35% greening, 60% growth |
| DBC3 (5 g/L Maltose + 10 g/L Sucrose) | 10% greening, 65% growth | 15-20% greening, 60% growth | 40% greening, 65% growth |
| DBC3 (5 g/L Maltose + 5 g/L Sucrose) | 15% greening, 35% growth | 15% greening, 30% growth | 35% greening, 65% growth |
| DBC3 no sugar | 0% greening, 5% growth | 0% greening, 5% growth | 2-5% greening, 10% growth |

% represents overall surface area of explant; % growth based on 100% Wildtype response on DBC3 with 30 g/L Maltose

TABLE 4
Media composition for sugar growth curves.

| | Amount/L | | | | | | | | |
|---|---|---|---|---|---|---|---|---|---|
| Components (stock conc.) | DBC3 | | | | | | | | |
| MS salts | 4.33 | g | | | | | | | |
| Thiamine (1 mg/ml) | 1 | ml | | | | | | | |
| Maltose | 30 | gm | 15 gm | 10 gm | 5 gm | 10 gm | 5 gm | 5 gm | 0 gm |
| Copper sulfate (0.05M) | 98 | uL | | | | | | | |
| Casein hydrolysate | | | | | | | | | |
| L-Proline | 0.69 | g | | | | | | | |
| myo-inositol | 0.25 | g | | | | | | | |
| Sucrose | | | | | | 5 gm | 10 gm | 5 gm | 0 gm |
| Gelrite | 2.5 | g | | | | | | | |
| Post Autoclave additives | | | | | | | | | |
| BAP (1 mg/ml) | 0.5 | ml | | | | | | | |
| 2,4-D (1 mg/ml) | 1 | ml | | | | | | | |
| pH with 1M KOH | 5.8 | | | | | | | | |

Components in Red are variable depending on treatment

As can be seen from Tables 3 and 4, reduction in the amount of maltose sugar below 30 g/L in the medium suppressed the greening of mutant callus in the light, as predicted. Optimal conditions for chloroplast transformation experiments could then be determined empirically. Selection of plastid transformants by complementation of the light-grown non-photosynthetic recipient

Conditions that maintained light-grown ppr10 mutant callus as pale-green or nearly albino when maintained on medium that requires photosynthesis were identified. Selection for restoration of photosynthetic competence after bombardment was in the light and using the same medium. In this case, plastid transformed lines were identified as green callus and regenerated green plants on medium that requires photosynthesis. Non-transformed tissues continued to grow as pale-green or nearly albino. Similarly, plant regeneration shortly after bombardment may be attempted. In this case, regenerated plants that were green would be easily identified from pale-green plants. It was anticipated that green transformed cells would grow faster and healthier than pale-green or albino cells, therefore transformed callus or plants were also expected to significantly outgrow non-transformed cells.

### Cloning of the Plastid-Expressed PPR10 Gene.

Nuclear-encoded proteins that are imported into chloroplasts carry an N-terminal extension called a chloroplast transit peptide (CTP) that is cleaved off of the protein after import into plastids. For expression in plastids, the nuclear-encoded PPR10 protein must be modified to remove the CTP. The PPR10 CTP can be predicted by a number of publicly available software applications including ChloroP and TargetP (Emanuelsson, 1999, 2000). Prediction identified the putative CTP cleavage site at amino acid position 37 of the PPR10 protein. After removal of the CTP, a new initiator methionine was included at the N-terminus along with an alanine in the second amino acid position, which is thought to be a stabilizing amino acid (Apel 2010) (SEQ ID NO:1).

In addition, for optimal protein expression in plastids, the synthetic ppr10 gene was created with plastid-preferred codons, which are significantly different from the codon usage used for nuclear-encoded genes. For codon optimization of the plastid version of the ppr10 gene, the AT-richness of the plastid genome was taken into consideration. Manual inspection of the nucleotide sequence of the nuclear-encoded ppr10 gene indicated numerous locations where a more AT-rich alternative codon could replace the native nuclear-encoded codon, resulting in the plastid codon optimized nucleotide sequence (SEQ ID NO:2) used for plastid transformation vectors.

### Additional Non-Photosynthetic Mutants for Complementation of the Plastid Photosynthetic Mutant Phenotype

### ATPC (ATP Synthase, Gamma Subunit)

Mutation in proteins that make up structural components of the photosynthetic apparatus or associated enzymes may also be good targets for photosynthetic complementation in the plastid. To this end, a mutant maize line that carried a non-photosynthetic mutation in the Zm-atpc gene was obtained. Similar to PPR10 above, the Zm-atpc mutation was maintained in heterozygous plants. Growth of heterozygous plants to maturity resulted in embryos that were segregating for the homozygous non-photosynthetic mutant phenotype. Immature embryos could be harvested in sterile tissue culture and homozygous callus lines identified by PCR using primers described below. Homozygous callus was then amplified in the dark on N6 medium or N6 medium with supplemental glucose for subsequent plastid transformation experiments. Alternatively, segregating seeds could be sown in soil, homozygous albino non-photosynthetic plants identified, and callus could be generated from hypocotyl sections in sterile tissue culture.

PCR analysis to identify homozygous atpc mutant callus was performed using the following primers:

ATPCF:
(SEQ ID NO: 23)
TTCGGGACAGGCTTGAAACT
ATPCR:
(SEQ ID NO: 24)
CCGTCTGGATCTCCTGGTTC
mu1:
(SEQ ID NO: 18)
5′-GCCTCCATTTCGTCGAATCCC

The ATPC gene was amplified using a mixture of ATPCF and ATPCR PCR primers. The ATPC gene that was disrupted by a Mu transposon was detected using a mixture of ATPCF and mu1 primers. The wild type ATPC gene genomic, cDNA and encoded protein sequence is set forth in SEQ ID NO:29, SEQ ID NO:30 (Maize ATPC genomic sequence available on the world wide web internet site “ncbi.nlm.nih.gov/gene/100284505): >NC_024465.2:c158180360-158179038 Zea mays cultivar B73 chromosome 7, B73 RefGen_v4, whole genome shotgun sequence SEQ ID NO:29) (ATP synthase subunit gamma, chloroplastic precursor [Zea mays] NP_001150872.1 ATP synthase subunit gamma, chloroplastic precursor [Zea mays] SEQ ID NO:30) and Maize ATPC cDNA sequence (SEQ ID NO:31).

Homozygous mutant callus was maintained in the dark. For selection of plastid transformants in the light, it was important to determine that mutant callus would not turn green in the light. Therefore, growth on media with reduced sugar (as above with PPR10) was performed. Optimal sugar content that prevents greening in the light was chosen and used in subsequent plastid transformation experiments.

### Cloning of the Plastid-Expressed ATPC Gene.

Similar to PPR10, the optimized plastid expressed ATPC gene was designed without its native chloroplast transit peptide (CTP), which was predicted to be cleaved at amino acid 35 of the nuclear-encoded protein. A new initiator methionine was included at the N-terminus along with an Alanine in the second amino acid position, which was thought to be a stabilizing amino acid (Apel 2010). Furthermore, the synthetic gene was created using plastid-preferred codons that are high in AT content.

### CHLI (Magnesium Chelatase Subunit I)

Mutations in genes involved in chlorophyll biosynthesis may also cause a non-photosynthetic phenotype. A mutant maize line was obtained that carried a non-photosynthetic mutation in the multi-subunit chlorophyll biosynthesis enzyme, magnesium chelatase. Similar to above, heterozygous seeds were obtained and sown in the greenhouse Immature embryos were harvested and used to generate callus in the dark on N6 medium. Homozygous callus was identified by PCR using primers and then amplified for subsequent plastid transformation experiments.

PCR analysis to identify homozygous chli mutant callus was performed using the following primers:

CHLIF:
(SEQ ID NO: 25)
AGGCCAACAGGGGCATACT
CHLIR:
(SEQ ID NO: 26)
CGCTGTCCCTTCCTTTCAAC
mu1:
(SEQ ID NO: 18)
5′-GCCTCCATTTCGTCGAATCCC

The CHLI gene was amplified for diagnostic analysis of heterozygous or homozygous mutant lines using a mixture of CHLIF and CHLIR PCR primers. The CHLI gene that was disrupted by a Mu transposon was detected using a mixture of CHLIF and mu1 primers. Heterozygous lines carry a mixture of wild-type CHLI gene and a disrupted Chli mutant gene, whereas homozygous mutants carry only the disrupted Chli gene. The maize CHLI genomic, protein, and cDNA sequence are set forth in SEQ ID NOS:32-34.

(Maize CHLI (also known as Oil Yellow): >NC_024468.2: c9166866-9165176 Zea mays cultivar B73 chromosome 10, B73 RefGen_v4, whole genome shotgun sequence, SEQ ID NO: 32) (>Maize CHLI amino acid sequence SEQ ID NO: 33) (>Maize CHLI cDNA sequence, SEQ ID NO: 34).

The same sugar sensitivity as above was used to identify medium where the homozygous mutant callus will not turn green when transferred to the light. Selection of plastid transformants was on this medium. The analysis showed that chli mutants will not turn green even on medium with high sugar levels.

### Cloning of the Plastid CHLI Gene

The CTP of the nuclear-encoded gene was predicted to be cleaved at amino acid 73. Similar to PPR10 and ATPC transgenes, the CTP was removed from the plastid-encoded CHLI transgene and a new methionine initiator and alanine as the second amino acid were included. Plastid-preferred codons that were high in AT content were also used in design of the synthetic gene.

### Example 2: Complementation of the Mutant Chli Gene in the Nucleus Confirms that Selection for Greening is Possible in Maize

Transformation of plant cells typically requires a selection scheme that kills cells that have not received transforming DNA while expression of the selectable marker taken up by transformed cells imparts resistance to an antibiotic or herbicide. Non-selected cells will die while cells with transforming DNA will multiply and grow in the presence of the selection agent. To test whether transformed cells can be identified solely by their green or greening phenotype, we first performed nuclear transformation of the chli mutant line using a cDNA of the wild-type Chli gene.

Embryogenic callus derived from immature embryos of homozygous chli mutant seeds were first identified by PCR and the inability to green on rich agar medium in the light. The callus was amplified by subculturing every 2 weeks until a large amount of callus was available for transformation experiments.

A cDNA for the full-length wild-type Chli gene (including its chloroplast transit peptide) was obtained from the University of Arizona and cloned into a nuclear expression cassette, between the maize Polyubiquitin 1 promoter and the CaMV 35S terminator to create plasmid pPTS57. Plasmid pPTS57 does not include any standard plant selectable marker gene.

Plasmid pPTS57 was bombarded into chli mutant callus using the PDS1000 bombardment process under standard conditions. Bombarded callus was incubated in the light on agar medium with 20% sugar at 28 C. Callus was subcultured every 2 weeks until green callus sectors were observed.

Two nuclear transformation experiments were conducted, with 2 bombarded plates in each experiment. In experiment 1, 6 green callus sectors were observed. In experiment 2, 1 green callus sector was observed. Green callus sectors were visible after about 6-8 weeks. It should be noted that all callus grew well on 20% sugar medium during this time, as expected. An example of green callus in the middle of non-transformed white callus is shown in FIG. 10. Green callus sectors were manually removed from surrounding non-green callus and subcultured for amplification of these nuclear transformed lines. These data indicate that selection for greening is possible, even in the absence of a lethal selectable agent to prevent growth of untransformed cells.

### Plastid Transformation Vectors for Complementation of Nuclear-Encoded Photosynthetic Mutant Recipient Lines

In some embodiments, plastid transformation vectors carry a region of homology to the plastid genome to direct integration of foreign transgenic DNA by homologous recombination, typically into an intergenic region of the plastid genome such that essential plastid functions are not disrupted. Homologous regions that direct transgene integration must be derived from the targeted plastid genome to maximize sequence identity. Regions of homology are usually ˜1-2 kb on both sides of the transgenic insert.

The maize chloroplast genome sequence (NC_001666.2) is available from GenBank and was used to retrieve all homologous sequences.

Initially, plastid transformation vectors will test targeting of transgenes to 2 different regions of the maize plastid genome.

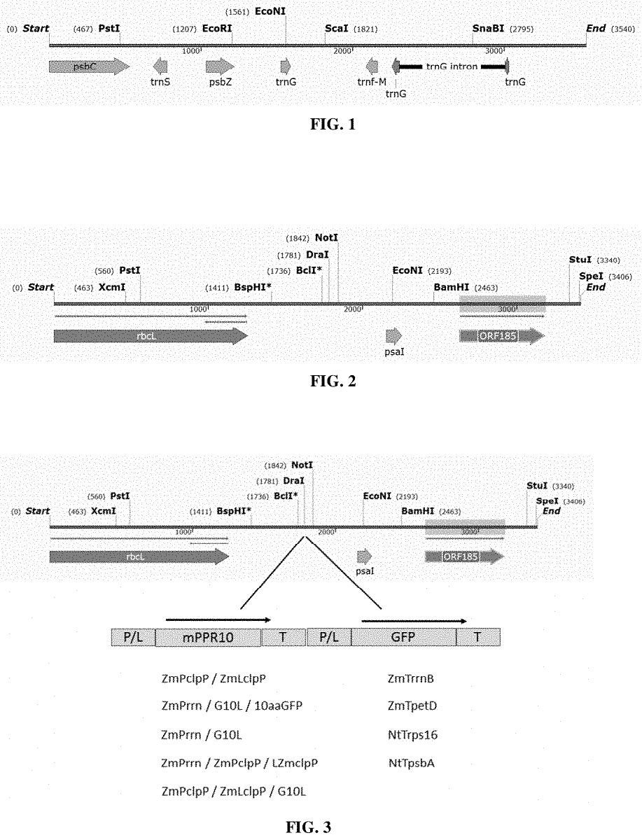

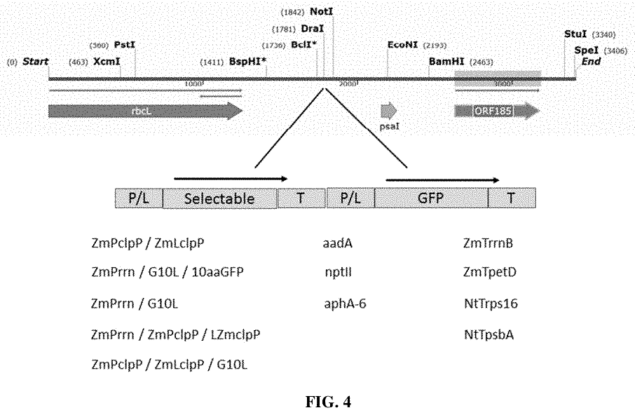

The Seal restriction enzyme site (FIG. 1) (psbC-trnG region; sequence #) served as a site of transgene integration. DraI or NotI restriction enzyme sites (FIG. 2) (rbcL region, sequence #) served as a site of transgene integration.

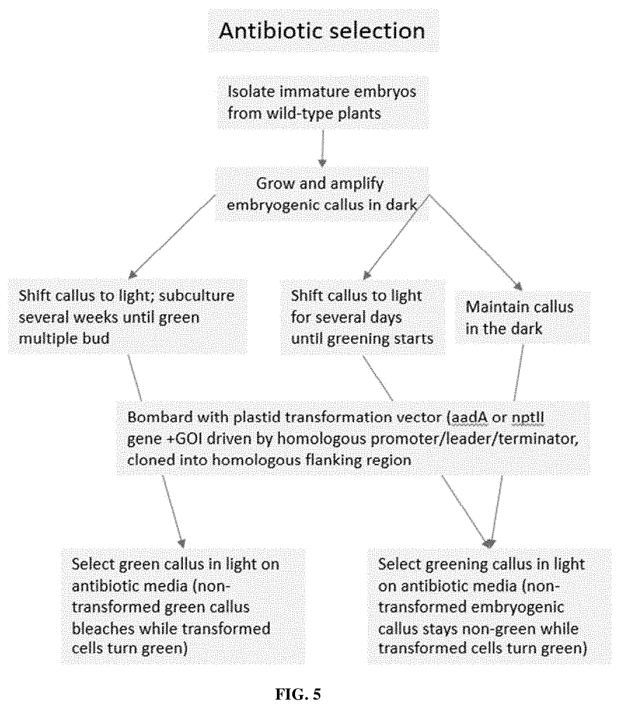

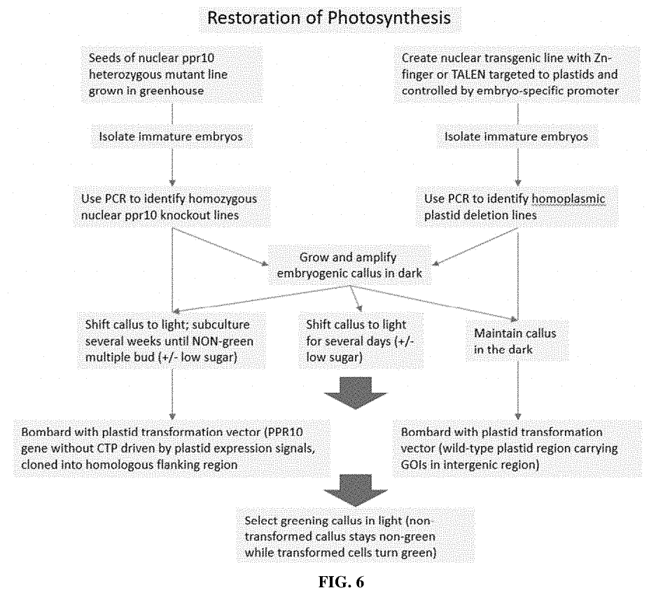

The promoter/leader sequences and transcript stability sequences provided in FIG. 3 were used to drive expression of the mature PPR10 coding sequence for expression in plastids and complementation of the non-photosynthetic phenotype, and GFP as a visual marker to help facilitate early identification of transformed lines. In subsequent experiments, GFP was replaced by trait genes of interest.

The plastid-encoded PPR10, ATPC, or CHLI genes were expressed from a variety of plastid gene or synthetic expression elements as shown in Table 5, with FIG. 3 as an example. These included plastid gene expression elements that have been shown to be expressed constitutively (for example, the promoter of the chloroplast ribosomal RNA operon, Prrn, with a synthetic translational leader derived from the bacteriophage T7 gene 10; G10L) or highly in non-green tissues (for example, the promoter, PclpP, and 5′-untranslated leader, LclpP, of the plastid-encoded clpP gene).

TABLE 5
Expression Elements for Expressing PPR10, ATPC, or CHLI genes.

| | Targeting | Flank | | | Gene 2 | Gene 2 | | Gene 2 |
|---|---|---|---|---|---|---|---|---|
| | species | Region | Gene 1 Promoter | Gene 1 | Terminator | Promoter | Gene 2 | Terminator |
| pPTS31 | maize | trnG/ycf9 | PZmClpP.LClpP.G10L | PPR10 | Trps16 | | | |
| pPTS32 | maize | trnG/ycf9 | PZmPrrnClpP.LClpP.G10L | PPR10 | Trps16 | | | |
| pPTS33 | maize | trnG/ycf9 | PZmClpP.LClpP.G10L | PPR10 | Trps16 | pZmPrrn.G10L | GFP | TNt.petD |
| pPTS34 | maize | trnG/ycf9 | PZmPrrnClpP.LClpP.G10L | PPR10 | Trps16 | pZmPrrn.G10L | GFP | TNt.petD |
| pPTS37 | maize | rbcL | PZmClpP.LClpP.G10L | PPR10 | Trps16 | pZmPrrn.G10L | GFP | TNt. petD |
| pPTS50 | maize | trnG/ycf9 | PZmPrrnClpP.LClpP.G10L | CHLI | TNt.rps16 | | | |
| | | | | (synthetic, | | | | |
| | | | | mature) | | | | |
| pPTS52 | maize | trnG/ycf9 | PZmPrrnG10L | CHLI | TNt.petD | | | |
| | | | | (synthetic, | | | | |
| | | | | mature) | | | | |
| pPTS64 | maize | trnG/ycf9 | PZMPrmnG10L + DB | CHLI | TNt.rps16 | | | |
| | | | | (synthetic, | | | | |
| | | | | mature) | | | | |
| pPTS48 | maize | trG/ycf9 | PZmPrrnG10L | ATPC | Ecoli | pZmPrrn.G10L | GFP | TNt.petD |
| | | | | (synthetic, | TrrnB | | | |
| | | | | mature) | | | | |
| pPTS49 | maize | rbcL | PZmPrrnG10L | ATPC | Ecoli | pZmPrrn.G10L | GFP | TNt.petD |
| | | | | (synthetic, | TrrnB | | | |
| | | | | mature) | | | | |
| pPTS51 | maize | trnG/ycf9 | PZmPrrnG10L | ATPC | Ecoli | | | |
| | | | | (synthetic, | TrrnB | | | |
| | | | | mature) | | | | |
| pPTS55 | maize | trnG/ycf9 | PZmPrrnClpP.LClpP.G10L | ATPC | Ecoli | | | |
| | | | | (synthetic, | TrrnB | | | |
| | | | | mature) | | | | |

The plastid transgenes also included a 3′-terminator sequence required to ensure mRNA stability of the plastid transgenes. The 3′-terminator may be derived from a known plastid gene, examples include the tobacco petD or rps16 genes. Alternatively, a terminator from the E. coli trrnB gene could be used.

In some embodiments, a fluorescent reporter gene, green fluorescent protein (GFP) also driven by plastid gene expression signals was included to help identify transplastomic tissues as they arise in tissue culture after transformation.

As an example, the selectable PPR10 gene and visual markers were cloned into the rbcL homology region, with the direction of transgene expression in the same orientation as the resident plastid genes.

### Example 3: Plant Transformation to Add Agronomically and/or Non-Agronomically Beneficial Traits

Non-photosynthetic callus derived from chloroplast deletion mutants or nuclear non-photosynthetic mutants would be used as recipient for plastid transformation using the complementing sequences as described above. The plastids of the non-photosynthetic mutant plant are then transformed with a transformation construct having a nucleic acid encoding a functional copy of the gene that was inactivated. Also present on the transformation construct, adjacent to the complementing sequence, is a nucleic acid encoding at least one gene conferring a beneficial trait, such as resistance to a herbicide, such as the gene for EPSPS that confers resistance to glyphosate, or resistance to a pesticide, such as a crystal protein gene from Bacillus thuringiensis that produces an insecticidal protein. The plant is then regenerated from the callus tissue by altering the growth culture conditions to induce shoot formation. Once the plant has grown to a sufficient size, it is transferred into soil. The insecticidal protein would be expressed in the plant tissue such that ingestion of the plant tissue by the target pest would result in death of the pest. Herbicide resistance would be verified by spraying with a lethal dose of glyphosate. Verification of the newly added agronomically and/or non-agronomically beneficial trait is done using PCR with primers specific for the newly added nucleic acid. Any agronomically and/or non-agronomically beneficial trait may be added to a plant as described herein.

### Example 4. Soybean Nuclear-Encoded Non-Photosynthetic Mutants

A few nuclear non-photosynthetic mutants and their causative genes have been identified in soybean, including mutation in the psbP gene encoding an extrinsic protein of Photosystem II that is critical for oxygen evolution during photosynthesis (Sandhu et al. 2016). Mutants in psbP are characterized as lethal-yellow and are maintained as heterozygous lines (Genetic Type Collection number T378H). Seeds of this line were obtained from the USDA and are grown to maturity in the greenhouse. Seeds are segregating green and yellow phenotypes. Yellow seedlings or embryos are homozygous mutants and can be used as recipients for chloroplast or nuclear transformation. The soybean PspB cDNA (SEQ ID NO:35 and protein sequence are set forth below.

>soybean psbP amino acid sequence
MANAIIMPGFLLRPTTQTTPFRLPNHTHSFHSPRCSLSLTPQSAKAFAVPR
RKALSLILSSYFLSEAGLALAQQSPVFREYIDAFDGYSFQYPGSWIQVRGA
GADIFFRDPFVLDENLSLEISSPSSSQYKSVEDLGPPQEAGKKVLKQYLTE
FMSTRLGVRRESNILSTSSRVADDGRLEEENDLRRIMDSFRVNKIAA*

Nuclear non-photosynthetic mutants in Chli1a and Chli1b paralogs in soybean also have been characterized. Mutation in the soybean chli 1a gene result in yellow viable plants, and include the Genetic Type Collection mutant line T219H and the commercially available MinnGold, whereas the CD-5 line is a mutant in the chli1b paralog. Various genomic, mRNA, and protein sequences of the soybean Chli1a and Chli1b genes are set forth below. Soybean CHLI1a genomic sequence>NC_038249.1:34356768-34359217 Glycine max cultivar Williams 82 chromosome 13, Glycine_max_v2.1, whole genome shotgun sequence is shown in SEQ ID NO:36 and Glycine max Mg-protoporphyrin IX chelatase subunit ChlI (CHLI), mRNA is SEQ ID NO:37.

SEQ ID NO:38 is >NP_001347251 magnesium-chelatase subunit ChlI, chloroplastic [Glycine max]; SEQ ID NO:39 is CHLI1b; GenBank: MG696843.1>MG696843 Glycine max cultivar Williams 82 chloroplast magnesium chelatase I subunit (ChlI1b) mRNA; and SEQ ID NO:40 is >Glycine max cultivar Williams 82 chloroplast magnesium chelatase I subunit (ChlI1b amino acid sequence).

Cotyledons from yellow embryos will be used to initiate embryogenic callus using methods known in the art (Finer 1988). In addition, yellow seedlings can be maintained in tissue culture media containing sugar and embryogenic axes will be used for transformation.

The psbP coding region will be codon-optimized for soybean chloroplast expression and the putative chloroplast transit peptide removed. The psbP gene sequence encoding the mature amino acid sequence will be driven by strong chloroplast gene expression signals, such as the chloroplast 16S ribosomal RNA operon promoter (Prrn) with the bacteriophage gene 10 translational leader (G10L), and a chloroplast 3′-untranslated region. The chimeric psbP gene will be cloned into a soybean chloroplast homologous flanking region to create a chloroplast transformation vector. After bombardment into soybean embryogenic callus or axis cultures, selection for chloroplast transformants will be for greening and active photosynthesis.

It may also be possible to use nuclear transformation to complement the non-photosynthetic psbP mutant and enhance photosynthetic performance Since expression of psbP is required for stabilization of Photosystem II in higher plants (Ifuku et al. 2005) and for high levels of oxygen evolution in Chlamydomonas (Mayfield et al. 1987), over-expression of psbP from the nucleus and subsequent targeting to the chloroplast may enhance oxygen evolving potential and stability of the Photosystem II apparatus under stress conditions.

### Example 4: Complementation of Nuclear Non-Photosynthetic Mutants Via Leaf Transformation

Overexpression of Babyboom (BBM) and Wuschel (WUS) transcription factors in maize and other monocots can induce embryogenesis from multiple tissues, including leaf tissues, that normally would not be embryogenic and thus not used for plant transformation. Use of leaf tissue for transformation has several advantages, including the availability of abundant tissue for transformation experiments and the relatively developed state of plastids in leaf tissue that may facilitate chloroplast transformation. Since nuclear nonphotosynthetic mutants can be challenging to maintain in tissue culture solely as callus, use of leaf tissue for plastid complementation of nuclear mutants also has the advantage of being able to create healthy, rapidly growing explants for transformation experiments.

Over-expression of BBM and WUS can be via stable nuclear transgenesis or via transient expression in the target tissue of choice. BBM and WUS gene expression can be controlled by a variety of transgene expression elements, though expression in leaf cells is preferred using either the maize ubiquitin 1 promoter or the maize PLTP gene promoter for ectopic expression of the BBM gene, whereas expression of WUS is often controlled by the IN2 gene promoter (GenBank: MT221179.1), an enhanced IN2 gene promoter variant, or controlled by an auxin inducible promoter, termed axi1G (Lowe et. al. 2018). Overexpression of BBM and WUS using these promoter combinations is sufficient for rapid somatic embryo formation from leaf base carrying rapidly dividing young plastids and more mature leaf tissues carrying developed chloroplasts.

The Chli homozygous mutant line described in [0199] above was used for stable nuclear re-transformation using plasmids that carry the PLTP:BBM transgene and auxin-inducible WUS (AxiG:WUS) transgene. In this case, Chli mutant callus was first allowed to regenerate into plants in tissue culture so that leaf base tissue could be collected for the retransformation experiments. Since leaf base tissue will not become embryogenic unless BBM and WUS transgenes are successfully introduced, nuclear retransformed lines are recovered simply after transformation of leaf base tissues by recovery of newly formed embryogenic callus. Mutant Chli callus was amplified in tissue culture and used for particle bombardment using the BBM and WUS plasmids. After ˜7-10 days, embryogenic callus formed on several leaf base sections, indicating successful introduction of BBM and WUS transgenes. Nuclear transgenic BBM+WUS lines in a chli mutant background [BBM/WUS(chli) lines] can be maintained as callus, as rapidly regenerating plantlets derived from callus or as callus derived from leaf base.

Leaf base tissues from the BBM/WUS (chli) mutant lines were then used for transformation of the wild-type Chli cDNA to select for green cells in culture, as a proof-of-concept for subsequent chloroplast transformation experiments. Numerous albino plants were regenerated from BBM/WUS (chli) mutant lines, leaf base tissue was isolated, and used for particle bombardment with the plasmid pPTS57 as described in [0210] above. In some experiments, selection was solely via observation of green cells in culture with no other herbicide or antibiotic selection, whereas in other experiments, selection for resistance to phosphinothricin on the pPTS57 plasmid was also used in addition to looking for green cells in culture.

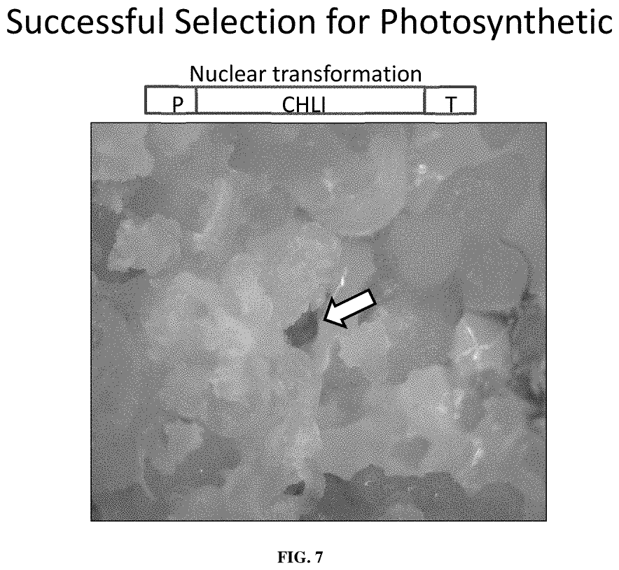

In multiple experiments using BBM/WUS (chli) mutant lines, multiple green calli similar to those seen previously and in FIG. 7 were observed, indicating complementation of the BBM/WUS (chli) mutant lines with a functional nuclear copy of the Chli transgene and restoration of photosynthesis. Green calli were dissected and amplified in tissue culture for further growth and plant regeneration. These experiments show that restoration of photosynthesis in leaf tissue from nuclear nonphotosynthetic mutants of monocots is possible.

The direct selection of green photosynthetically competent calli described above is in contrast to the statements of Hajj et al. (2018) that indicates, “In higher plants, direct selection for restoration of photosynthesis is not possible. This is because early in the transformation process cells and tissues are propagated on media containing sucrose. This allows both transformed cells and nontransformed mutant cells to proliferate during the early stages of transformation. Following regeneration, nontransformed shoots vastly outnumber transplastomic shoots hindering their identification. Selection for photosynthesis is possible once shoots are moved to media lacking sucrose, which is difficult to achieve with large numbers of shoots in vitro.” It should be noted that media containing sucrose was used in the Applicant's experiments described herein and green calli were readily observed, indicating that direct selection for restoration of photosynthesis is indeed possible.

### Example: Nuclear Co-Transformation of BBM/WUS (Chli) Lines

For nuclear cotransformation of BBM/WUS (chli) line, albino callus was first amplified on CHLI-CIM media (Table 6) for 6 weeks, then co-bombarded with plasmids pPTS321+pPTS129 (plasmid maps FIG. 1 and FIG. 2) on CHLI-CIM osmotic medium (Table 7) for several hours prior to bombardment. Bombardment conditions utilizing the Bio-Rad PDS-1000/He Biolistic Particle Delivery System were at 1100 psi using 0.4 micron gold particles coated with equal amounts (10 ug each) of plasmids pPTS321+pPTS129. Plasmid PTS129 plasmid carries a ZsGreen fluorescent reporter gene driven by the maize Ubiquitin 1 promoter, a chloroplast transit peptide for targeting the protein to plastids and the CaMV 35S terminator, whereas PTS321 carries the native CHLI cDNA driven by the same maize ubiquitin promoter and CaMv 35S terminator, and carries a hygromycin resistance gene used for selection of nuclear transformants. The CHLI cDNA carries its own native chloroplast transit peptide to deliver the protein to chloroplasts.

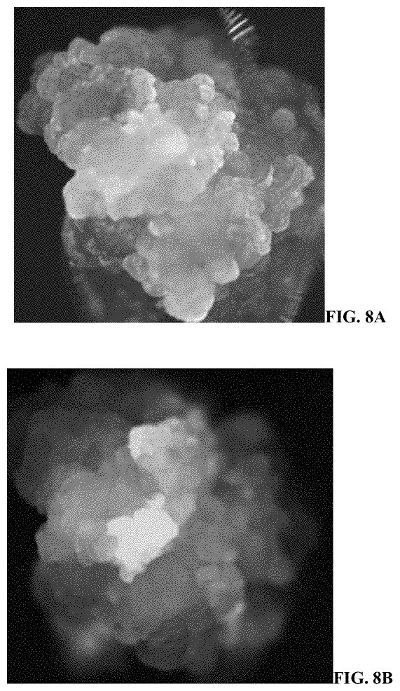

Bombarded plates were kept in dark chamber at 25° C. overnight. After 24 hours, bombarded explants were transferred on MS3 medium (Table 8) with 50 mg/L Hygromycin and transferred in a growth chamber under light (16 h light, 8 h dark, light intensity 28μ mole, temperature 25° C.). Explants were sub-cultured on fresh MS3+Hygromycin 50 mg/L every two weeks. From a total of 130 calli transformed with pPTS321+pPTS129 (one callus=one explant) 50 callus lines were selected that also express ZsGreen after the second cycle of selection on Hygromycin 50 mg/L. Out of 50 calli 33 were transferred on MS3+50 mg/L Hygromycin for a third round of selection to allow the transformed callus clusters to multiply and grow. Numerous ZsGreen positive calli developed green (chlorophyll-containing) sectors within one week after transfer to second round of selection (FIG. 8) indicating complementation of the chli mutation and are currently regenerating into green plants. Successful nuclear complementation of the chli mutant and cotransformation with ZsGreen expands the utility of our approach to simultaneous introduction of multiple transgenes, all using greening complementation as the selection system.

TABLE 6
CHLI callus induction media (CIM)

| | | |
|---|---|---|
| Chu N6 salts | 4 | g |
| L -Proline | 2.88 | g |
| 2,4-D-1 mg/L | 1 | ml |
| Casein Hydrolysate- 0.1 g/L | 0.1 | g |
| MES | 0.5 | g |
| Sucrose | 30 | g |
| Gelzan | 2.5 | g |
| Erickson Vitamins-1 ml of 1000x stock | 1 | ml |
| solution | | |
| Thiamine-625 ml/L (stock 0.8 mg/ml) | 625 | ul |
| Silver Nitrate - 0.1. ml/L (stock 8.5 mg/L) | 100 | ul |

TABLE 7
CHLI CIM osmotic media

| | | |
|---|---|---|
| Chu N6 salts | 4 | g |
| L -Proline | 2.88 | g |
| 2,4-D-1 mg/L | 1 | ml |
| Casein Hydrolysate- 0.1 g/L | 0.1 | g |
| MES- 0.5 g/L | 0.5 | g |
| Mannitol | 36.4 | g |
| Sorbitol | 36.4 | g |
| Gelzan- 2.5 g/L | 2.5 | g |
| Erickson Vitamins-1 ml of 1000x stock | 1 | ml |
| solution | | |
| Thiamine-625 ml/L (stock 0.8 mg/ml) | 625 | ul |
| Silver Nitrate - 0.1. ml/L (stock 8.5 mg/L) | 100 | ul |

TABLE 8
MS3 media

| | | |
|---|---|---|
| MS salts | 4.33 | g |
| MS Vitamins | 1 | ml |
| Maltose 40 g/L | 40 | g |
| Casein Hydrolysate- 0.5 g/L | 0.5 | g |
| MES- 1.95 g/L | 1.95 | g |
| Picloram- 2.2. mg/L | 2.2 | mg |
| BAP-1 mg/ml | 1 | ml |
| Ascorbic acid- 1.0 mg/L | 1 | ml |

Although the present disclosure has been described with reference to preferred embodiments, workers skilled in the art will recognize that changes may be made in form and detail without departing from the spirit and scope of the disclosure.

What is claimed is:

1. A method for transforming a plant plastid with a DNA molecule comprising:
(a) introducing at least one DNA molecule comprising a first DNA sequence into a recipient non-photosynthetic plant cell comprising a loss-of-function mutation in each copy of a nuclear gene encoding a chloroplast-localized protein required for photosynthesis, wherein the DNA molecule encodes a protein having an enzymatic and/or biological activity of the chloroplast-localized protein required for photosynthesis;
(b) exposing the transformed plant cell from step (a) to light sufficient to support greening of a photosynthetic plant cell; and,
(c) selecting a green photosynthetic plant cell comprising a transformed plant plastid containing a plastid genome comprising the DNA molecule from the plant cells exposed to the light in step (b), thereby transforming a plant plastid with a DNA molecule.

2. The method of claim 1, wherein the non-photosynthetic cell is homozygous for the loss of-function mutation in the nuclear gene.

3. The method of claim 1, wherein the protein having an enzymatic and/or biological activity of the chloroplast-localized protein provides for improved photosynthesis, an increased accumulation of the protein encoded by the nuclear gene, a decrease in the Km of the protein encoded by the nuclear gene for a substrate of the protein, an increase in the Kcat of the protein encoded by the nuclear gene, or any combination thereof.

4. The method of claim 1, wherein a selectable antibiotic resistance- or herbicide resistance-conferring gene is absent from the DNA molecule.

5. The method of claim 1, further comprising the step of obtaining a transplastomic plant comprising the transformed plant plastids from the green photosynthetic plant cell of step (c).

6. The method of claim 5, further comprising the step of selecting a homoplasmic transplastomic plant comprising the transformed plant plastids from the transplastomic plant.

7. The method of claim 1, wherein the DNA molecule comprises: (i) a promoter which is functional in plant plastids; (ii) DNA encoding the protein having an enzymatic and/or biological activity of the chloroplast-localized protein, and (iii) a DNA comprising a transcription terminator and/or DNA which encodes an mRNA stability element; wherein the promoter, the DNA encoding the protein, and the DNA comprising a transcription terminator and/or DNA which encodes an mRNA stability element are operably linked.

8. The method of claim 1, wherein the DNA molecule further comprises DNA homologous to a target insertion site in the plastid genome.

9. The method of claim 1, wherein the green photosynthetic plant cell is further selected for insertion of the DNA molecule comprising DNA encoding the protein having an enzymatic and/or biological activity of the chloroplast-localized protein into a target insertion site in the plastid genome.

10. The method of claim 1, wherein a selectable antibiotic resistance- or herbicide resistance-conferring gene is not introduced in the recipient plant cell in step (a) and/or wherein a selection for antibiotic resistance or herbicide resistance is not performed.

11. The method of claim 1, wherein: (i) a second heterologous DNA molecule is introduced with the DNA molecule; or (ii) wherein the DNA molecule comprises or further comprises a heterologous DNA molecule.

12. The method of claim 11, wherein the heterologous DNA molecule confers an agronomically beneficial trait, a desirable non-agronomic trait, or combination thereof.

13. The method of claim 11, wherein the plastid genome comprises an insertion of the heterologous DNA molecule at a location which is not immediately adjacent to the DNA molecule.

14. The method of claim 1, wherein the recipient homoplasmic non-photosynthetic plant cells in step (a) are grown in culture as callus, embryogenic callus, organogenic cultures, suspension cells, or are leaf cells.

15. The method of claim 1, further comprising the step of regenerating a transplastomic plant from the green photosynthetic plant cell of step (c).

16. The method of claim 1, wherein the recipient homoplasmic non-photosynthetic plant cells in step (a) are located in a whole plant, a whole plant seedling, or a whole plant part.

17. The method of claim 1, wherein the selecting of plant cells in step (c) comprises selecting a sector of green plant cells.

18. The method of claim 1, wherein the plant cell is a monocot plant cell or wherein the plant cell is a maize, sorghum, wheat, or rice plant cell.

19. The method of claim 18, wherein the nuclear gene or wild-type nuclear gene is a PPR10, ATPC, CHLI gene, an orthologue thereof, or paralog thereof.

20. The method of claim 18, wherein the nuclear gene comprising the loss-of-function mutations is a non-photosynthetic gene.

21. The method of claim 20, wherein the nuclear gene comprising the loss-of-function mutation is a ppr10 gene, an atpc gene, or a chli gene.

22. The method of claim 1, wherein the plant cell is a dicot plant cell.

23. The method of claim 22 wherein the dicot plant cell is a cotton, soybean, Brassica sp., potato, or tomato plant cell.

24. The method of claim 22, wherein the nuclear gene or wild-type nuclear gene is a CHLI gene, a PsbP gene, an orthologue thereof, or a paralog thereof.

25. The method of claim 22, wherein the plant cell is a soybean plant cell and the nuclear gene or wild-type nuclear gene is a CHLI 1a gene, a CHLI 1b gene, or a GmPsbP gene.

26. The method of claim 22, wherein the plant cell is a soybean plant cell and the nuclear gene comprising the loss-of-function mutations is a chli 1a gene, a chli 1b gene, or a psbp gene.

27. The method of claim 22, wherein the plant cell is not a tobacco or Arabidopsis plant cell.

28. The method of claim 1, wherein a nucleic acid that provides for expression of a morphogenetic gene that allows for regeneration from a transformed sector is introduced into the recipient non-photosynthetic plant cell or plant and is expressed.

29. The method of claim 1 wherein a nucleic acid that provides for expression of Babyboom (BBM) and/or a Wuschel (WUS) polypeptide is introduced into the recipient non-photosynthetic plant cell or plant and is expressed.

30. A method for transforming a plant plastid with a DNA molecule comprising:
(a) introducing at least one DNA molecule comprising a first DNA sequence into a recipient non-photosynthetic plant cell comprising a loss-of-function mutation in each copy of a nuclear gene encoding a chloroplast-localized protein required for photosynthesis, wherein the DNA molecule encodes a protein having an enzymatic and/or biological activity of the chloroplast-localized protein required for photosynthesis;
(b) exposing the transformed plant cell from step (a) to light sufficient to support greening of a photosynthetic plant cell; and,
(c) selecting a sector of green photosynthetic plant cells comprising a transformed plant plastid containing a plastid genome comprising the DNA molecule from the plant cells exposed to the light in step (b), thereby transforming a plant plastid with a DNA molecule.
(d) introducing a nucleic acid that provides for expression of a morphogenetic gene that allows for regeneration from a transformed sector into the recipient non-photosynthetic plant cell or plant and is expressed.

31. The method of claim 30 and (d) introducing a nucleic acid that provides for expression of a morphogenetic gene that allows for regeneration from a transformed sector into the recipient non-photosynthetic plant cell or plant and is expressed.

32. The method of claim 30 wherein the nucleic acid that provides for expression of Babyboom (BBM) and/or a Wuschel (WUS) polypeptide is introduced into the recipient non-photosynthetic plant cell or plant and is expressed.

33. A method of expressing a morphogenetic gene in a nuclear-transformed cell or sector to enable multiplying and/or regenerating the cell.

34. The method of claim 33 wherein the nuclear-transformed cell or sector is derived from an immature embryo, embryogenic callus, an organogenic callus, a meristem or a leaf.

35. The method of claim 33 and introducing the morphogenic gene into the nuclear-transformed cell or sector prior to or after the identification of the nuclear transformed cell or sector.

36. The method of claim 33 wherein the nuclear-transformed cell or sector is placed on a medium containing plant growth regulators sufficient to enable cell multiplication or regeneration.

37. The method of claim 33 wherein the morphogenetic gene is a Babyboom (BBM) and/or a Wuschel (WUS) polypeptide.

38. The method of claim 33 wherein the nuclear-transformed cell is a monocot or dicot cell.
